(19) United States
(12) Patent Application Publication
(10) Pub. No.: US 2026/0224913 A1
(43) Pub. Date: Aug. 6, 2026

(54) METHODS FOR PLAN ADJUSTMENT OF RADIATION THERAPY, RADIATION THERAPY SYSTEMS, AND RELATED DEVICES

(71) Applicant: MEVION MEDICAL EQUIPMENT CO., LTD., Suzhou (CN)

(72) Inventors: Tianbao ZHU, Suzhou (CN); Pu YANG, Suzhou (CN); Zixu ZHAO, Suzhou (CN); Guihua LI, Suzhou (CN)

(73) Assignee: MEVION MEDICAL EQUIPMENT CO., LTD., Suzhou (CN)

(21) Appl. No.: 19/634,025

(22) Filed: Mar. 30, 2026

Related U.S. Application Data
(63) Continuation-in-part of application No. PCT/CN2024/124754, filed on Oct. 14, 2024.

(30) Foreign Application Priority Data
Oct. 18, 2023 (CN) 202311352360.3

(51) Int. Cl. A61N 5/10

(52) U.S. Cl. CPC A61N 5/1031; A61N 5/1038; A61N 5/1039; A61N 2005/1034

(57) ABSTRACT

The present disclosure relates to a method for plan adjustment of radiation therapy, a radiation therapy system, and related devices. The method includes: obtaining simulation CT information and treatment plan information of a patient. The method includes: in a current radiation therapy corresponding to the treatment plan information, obtaining surface optical image information of an irradiated region of the patient. The method includes: obtaining irradiation dose information of the target volume and the organ-at-risk region. The method includes: according to the irradiation dose information of the target volume and the organ-at-risk region and the treatment plan information, determining whether treatment plan information of a subsequent irradiation fractionation needs to be adjusted. The method includes: in response to a determination that the treatment plan information of the subsequent irradiation fractionation needs to be adjusted, according to a dose distribution of the irradiated region and a treatment prescription corresponding to the patient, adjusting the treatment plan information of the subsequent irradiation fractionation.

### CROSS-REFERENCE TO RELATED APPLICATIONS

This application is a Continuation-in-part of International Application No. PCT/CN2024/124754, filed on Oct. 14, 2024, which claims priority to Chinese Patent Application No. 202311352360.3, filed on Oct. 18, 2023, the entire contents of each of which are hereby incorporated by reference.

### TECHNICAL FIELD

The present disclosure generally relates to a field of radiation therapy, and in particular, to a method for plan adjustment of radiation therapy, a radiation therapy system, and a related device.

### BACKGROUND

In conventional radiation therapy, the radiation therapy is typically executed by delineating a target volume and an organ-at-risk region based on a set of simulation positioning images of a patient, creating a radiation therapy plan, and executing radiation irradiation. During a treatment process, it is typically idealized that anatomical structure and a treatment position of the patient remain unchanged relative to a dose delivery system of the radiation therapy. Similarly, a fixed prescription dose is typically used during the treatment process, if biological response of tissues to a dose remains unchanged during the treatment process. However, from a long-term perspective, there are many variable factors during the treatment process, and the variable factors affect the effect of radiation therapy. Therefore, adaptive radiation therapy has received significant attention. During an adaptive radiation therapy process, a doctor may adjust a treatment plan based on factors such as organ motion within a fractionation treatment, changes in the target volume between fractionation treatments, and biological effects, so as to execute the radiation therapy more accurately and effectively.

Taking proton therapy as an example, the proton therapy, as an advanced radiation therapy technology for “targeted destruction” of lesions, combined with an adaptive therapy technology, achieves better treatment effects. Quality assurance is an important means for accurate proton therapy. Current plan adjustment methods employ a water phantom for simulation, and a simulation verification result cannot accurately reflect a condition during treatment, thereby affecting the accuracy of plan adjustment for radiation therapy.

Based on the above, the present disclosure provides a method for plan adjustment of radiation therapy, a radiation therapy system, and related devices to improve related technologies.

### SUMMARY

An objective of the present disclosure is to provide the method for plan adjustment of radiation therapy, the radiation therapy system, and the related devices. The method for plan adjustment of radiation therapy, the radiation therapy system, and the related devices accurately acquire irradiation dose information for indicating an irradiation dose of a target volume and an organ-at-risk region of a patient by detecting Cherenkov radiation of the patient. The method for plan adjustment of radiation therapy, the radiation therapy system, and the related devices determine a quality assurance result of a treatment plan by comparing acquired actual measurement data with theoretical calculation data. The method for plan adjustment of radiation therapy, the radiation therapy system, and the related devices solve a problem of low accuracy of plan adjustment of radiation therapy in a water phantom simulation mode.

The objective of the present disclosure is achieved by the following technical solutions:

The present disclosure provides the method for plan adjustment of radiation therapy, the method including the following steps.

S1, obtaining simulation computed tomography (CT) information and treatment plan information of a patient, the simulation CT information includes a simulation CT image and region segmentation information corresponding to the simulation CT image, the region segmentation information is used to segment a target volume and an organ-at-risk region on the simulation CT image, and the treatment plan information includes a treatment plan parameter of the target volume and the organ-at-risk region.

S2, in a current fractionation of radiation therapy corresponding to the treatment plan information, obtaining surface optical image information of an irradiated region of the patient by measuring Cherenkov radiation emitted from the patient due to particle irradiation.

S3, obtaining a dose distribution of the irradiated region based on the surface optical image information, the simulation CT information, and a preset mapping relationship, and obtaining irradiation dose information of the target volume and the organ-at-risk region based on the dose distribution of the irradiated region.

S4, determining whether the treatment plan information for a subsequent fractionation of irradiation needs to be adjusted based on the irradiation dose information and the treatment plan information.

S5, in response to determining that the treatment plan information for the subsequent fractionation of irradiation needs to be adjusted, adjusting the treatment plan information for the subsequent fractionation of irradiation based on the dose distribution of the irradiated region and a treatment prescription corresponding to the patient.

In a second aspect, the present disclosure provides an electronic device, the electronic device comprising a memory and one or more processors, the memory storing a computer program, the one or more processors being configured to implement the following steps in response to executing the computer program:

S1, obtaining simulation CT information and treatment plan information of a patient, the simulation CT information includes a simulation CT image and region segmentation information corresponding to the simulation CT image, the region segmentation information is used to segment a target volume and an organ-at-risk region on the simulation CT image, and the treatment plan information includes a treatment plan parameter of the target volume and the organ-at-risk region.

S2, in a current fractionation of radiation therapy corresponding to the treatment plan information, obtaining surface optical image information of an irradiated region of the patient by measuring Cherenkov radiation emitted from the patient due to particle irradiation.

S3, obtaining a dose distribution of the irradiated region based on the surface optical image information, the simulation CT information, and a preset mapping relationship, and obtaining irradiation dose information of the target volume and the organ-at-risk region based on the dose distribution of the irradiated region.

S4, determining whether the treatment plan information for a subsequent fractionation of irradiation needs to be adjusted based on the irradiation dose information and the treatment plan information.

S5, in response to determining that the treatment plan information for the subsequent fractionation of irradiation needs to be adjusted, adjusting the treatment plan information for the subsequent fractionation of irradiation based on the dose distribution of the irradiated region and a treatment prescription corresponding to the patient.

In a third aspect, the present disclosure provides a radiation therapy system.

The radiation therapy system includes the electronic device according to the present disclosure.

The radiation therapy system includes a dose acquisition device, configured to obtain surface optical image information of an irradiated region of a patient.

The radiation therapy system includes a radiation device, configured to perform particle irradiation on a treatment region of the patient.

In a fourth aspect, the present disclosure provides a computer-readable storage medium. The computer-readable storage medium stores a computer program. The computer program implements the steps of any one of the methods or implements the functions of any one of the electronic devices when executed by one or more processors.

In a fifth aspect, the present disclosure provides a computer program product. The computer program product includes a computer program. The computer program implements the steps of any one of the methods or implements the functions of any one of the electronic devices when executed by one or more processors.

### BRIEF DESCRIPTION OF THE DRAWINGS

The present disclosure is further described below in conjunction with the accompanying drawings and specific embodiments.

FIG. 1 is a schematic flowchart of a method for plan adjustment of radiation therapy according to some embodiments of the present disclosure.

FIG. 2 is a flowchart of an exemplary process for image error detection according to some embodiments of the present disclosure.

FIG. 3 is a flowchart of an exemplary process for laser error detection according to some embodiments of the present disclosure.

FIG. 4 is a flowchart of an exemplary process for acquiring surface optical image information according to some embodiments of the present disclosure.

FIG. 5 is a flowchart of an exemplary process for correcting body surface optical information according to some embodiments of the present disclosure.

FIG. 6 is a flowchart of an exemplary process for generating a correction parameter according to some embodiments of the present disclosure.

FIG. 7 is a schematic diagram of a camera angle according to some embodiments of the present disclosure.

FIG. 8 is a flowchart of an exemplary process for determining whether skin hyperemia exists according to some embodiments of the present disclosure.

FIG. 9 is a block diagram of a structure of an electronic device according to some embodiments of the present disclosure.

FIG. 10 is a schematic diagram of a structure of a radiation therapy system according to some embodiments of the present disclosure.

FIG. 11 is a schematic diagram of a structure of a computer program product according to some embodiments of the present disclosure.

### DETAILED DESCRIPTION

Technical solutions in embodiments of the present disclosure are described below with reference to the accompanying drawings in the embodiments of the present disclosure. The described embodiments are merely some rather than all the embodiments of the present disclosure. Those skilled in the art may understand that, with the development of technology and the emergence of new scenarios, the technical solutions provided in the embodiments of the present disclosure are also applicable to similar technical problems.

It should be noted that, on the premise of no conflict, the following described embodiments or technical features may be arbitrarily combined to form new embodiments.

In the present disclosure, terms such as “exemplary” or “for example” are used to represent examples, illustrations, or explanations. Any embodiment or design scheme described as “exemplary” or “for example” in the present disclosure should not be construed as being more preferred or advantageous than other embodiments or design schemes. Rather, the use of terms such as “exemplary” or “for example” is intended to present related concepts in a specific manner.

Descriptions such as “first” and “second” appearing in the embodiments of the present disclosure are used only for illustration and distinguishing of described objects, have no order, do not indicate any special limitation on the number of devices in the embodiments of the present disclosure, and cannot constitute any limitation on the embodiments of the present disclosure.

The technical field and related terms of the embodiments of the present disclosure are briefly described below.

The present disclosure provides a method for plan adjustment of radiation therapy, a radiation therapy system, and related devices. In the embodiments of the present disclosure, particles for radiation therapy may include protons, heavy ions, electrons, X-rays, gamma rays, or the like. These particles have different energies and masses and may be used for targeted radiation against tumors.

In the embodiments of the present disclosure, the particles themselves are charged particles (e.g., the protons, etc.), or the particles irradiate a human body may generate charged particles (e.g., secondary electrons, etc.).

Proton therapy is a radiation therapy technique that uses a high-energy proton beam to precisely treat tumors. Compared with conventional X-ray radiotherapy, the proton therapy may better control delivery of a radiation dose, thereby reducing damage to organ-at-risk regions, and improving treatment effects.

A principle of proton therapy is to use a physical characteristic of the protons, i.e., after entering the human body, the proton beam reaches a maximum dose (Bragg peak) at a certain depth, and then sharply decreases until it stops. This characteristic enables the proton beam to release a maximum dose within the tumor while reducing dose deposition in organ-at-risk regions behind the tumor, thereby reducing side effects caused by the treatment. The proton therapy is applicable to various types of tumors, including pediatric tumors, cranial tumors, head and neck tumors, thoracic tumors, abdominal tumors, bone and soft tissue tumors, etc. The proton therapy is particularly suitable for tumors around critical organs or tumors sensitive to radiation. Compared with conventional radiotherapy, the proton therapy may better protect the organ-at-risk regions and the organs, reducing side effects caused by the treatment. Especially for pediatric patients, the proton therapy may reduce long-term treatment sequelae and lower a risk of secondary tumors. Some studies indicate that the proton therapy may provide treatment effects comparable to conventional radiotherapy in some cases while reducing adverse reactions.

Cherenkov radiation (CR) refers to a luminescence phenomenon generated when a charged particle moves in a medium at a speed exceeding the speed of light in the medium, and an essence of the Cherenkov radiation is polarization of atoms or molecules in the medium caused by the charged particle passing through the medium. For example, after the human body is irradiated by the protons, a visible light radiation phenomenon occurs during a treatment process, and a light field may be detected.

An optical measurement device may be used to detect the light field of the Cherenkov radiation. For example, the optical measurement device may include a light detector configured to detect Cherenkov radiation from the patient and/or a transparent photoelectric medium. The transparent photoelectric medium may be a gas, a liquid, or a solid. A thickness or volume of the human body and the transparent photoelectric medium is different, and corresponding Cherenkov radiation also changes accordingly. A count of the light detector may be one or more. A plurality of light detectors may be spaced apart to form a light detector array. In a case where the patient may block a field of view of a single light detector (e.g., a particle radiation source and the transparent photoelectric medium are located above the patient, and the light detector is located below the patient), other light detectors may still detect the Cherenkov radiation. The transparent photoelectric medium may be positioned such that any Cherenkov radiation generated by the transparent photoelectric medium is not blocked by the patient. For example, the transparent photoelectric medium and the light detector may be arranged to be positioned closer to the particle radiation source than the patient. The light detector may be an optical camera, a closed-circuit television, a video camera, etc. In some embodiments, the light detector is also used for photographing, recording, or monitoring the patient or another object. The other object may be, for example, a human tissue-simulating phantom, a water phantom, a treatment couch, a treatment instrument, or the like.

The particle radiation source may be mounted on a rotatable gantry, and the gantry may rotate around the patient or the treatment couch. In some embodiments, the light detector may be mounted on the gantry and may rotate together with the particle radiation source. During radiation therapy or a calibration process, the particle radiation source rotates around the patient or the treatment couch.

A particle dose on a surface of the human body refers to particle dose deposition on the surface of the human body during a treatment process. For example, a unit of the particle dose is Gray (Gy), milligray (mGy), etc.

Quality assurance is an important means for precise proton therapy. An implementation of related quality assurance is to replicate a treatment plan on a water phantom, and compare a dose distribution in the water phantom measured by an external dose measurement device with a dose distribution in the water phantom calculated by a treatment planning system, thereby verifying accuracy of dose delivery of the treatment plan.

The water phantom refers to a phantom formed with water or purified water as the main imaging medium, and the water phantom may also be filled with auxiliary imaging media such as air, metal wires, or the like. A dose distribution in the water phantom differs from a dose distribution in the human body. It may be understood that using the water phantom for simulation is a static manner. The static manner does not capture actual internal conditions of a simulated patient. For example, the static manner does not simulate biological changes of the patient, organ motion, or actual positional changes of a target volume.

Therefore, a method for plan adjustment of radiation therapy based on the water phantom does not account for individual differences of the patient, and does not accurately reflect conditions during treatment, thereby affecting accuracy of the radiation therapy.

Based on the above, the present disclosure provides a method for plan adjustment of radiation therapy, a radiation therapy system, and related devices to improve related technologies. A direct purpose of the method for plan adjustment of the present disclosure is not to obtain a diagnostic result or a health status, nor is the direct purpose for therapeutic purposes. The method for plan adjustment merely performs data processing on treatment plan information of the patient to provide a basis for a doctor to select a further plan. The method for plan adjustment facilitates the doctor to make an effective judgment for further work.

The method for plan adjustment provided by embodiments of the present disclosure may run on an electronic device. The electronic device may be a device with computing capability, such as a computer, a server (including a cloud server), or the like. The solution provided by embodiments of the present disclosure is specifically described through the following embodiments.

### Embodiment of the Method for Plan Adjustment

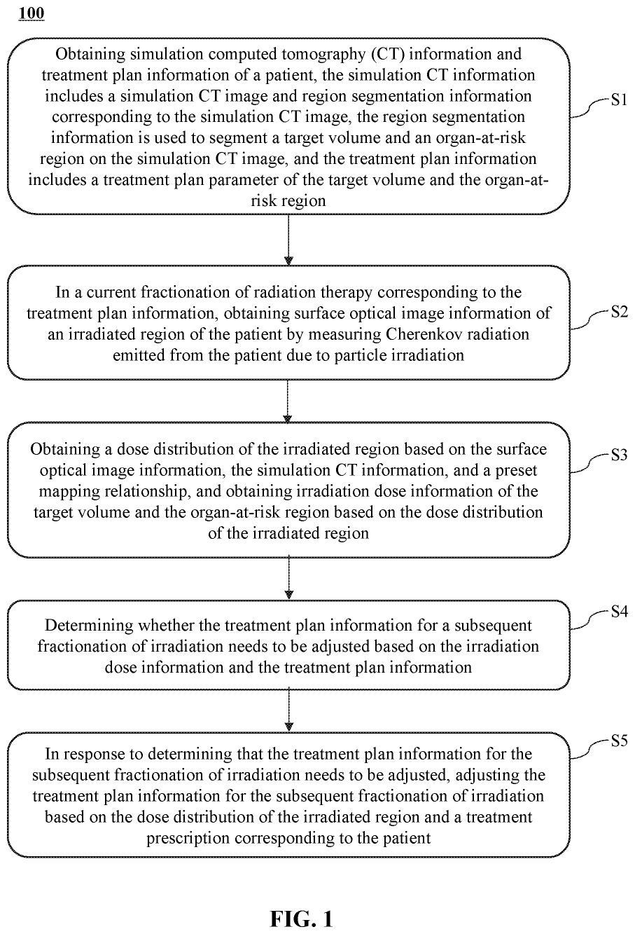

As shown in FIG. 1, FIG. 1 is a schematic flowchart of a method for plan adjustment of the radiation therapy according to some embodiments of the present disclosure. In some embodiments, the process 100 may be executed based on a processor of the electronic device. As shown in FIG. 1, the process 100 includes the following steps:

In S1, obtaining simulation computed tomography (CT) information and treatment plan information of a patient. The simulation CT information includes a simulation CT image and corresponding region segmentation information. The region segmentation information is used to segment a target volume and an organ-at-risk region on the simulation CT image. The treatment plan information includes a treatment plan parameter of the target volume and the organ-at-risk region.

The simulation CT information refers to a data set specifically acquired for the radiation therapy, the data set includes a CT image of the patient and a target volume and an organ-at-risk region determined on the CT image. Positions (regions) of the target volume and the organ-at-risk region may be determined manually or by using a computer to automatically perform region segmentation.

The target volume refers to a region requiring the radiation therapy. For example, the target volume may include a tumor lesion and surrounding tissues infiltrated by the tumor lesion.

The organ-at-risk region refers to normal tissue structures adjacent to the target volume. For example, when the target volume is a lung, the organ-at-risk region may include a heart, an esophagus, a spinal cord, or the like.

The simulation CT image refers to a medical imaging image used for radiation therapy plan formulation and simulation positioning. In some embodiments, the simulation CT image may be obtained through computed tomography scanning.

The region segmentation information refers to data indicating positions of the target volume and the organ-at-risk region on the simulation CT image. In some embodiments, the region segmentation information may include labels of one or more target volumes and organ-at-risk regions, and coordinate value sets thereof. A label is configured to indicate which target volume or organ-at-risk region the label corresponds to. The processor may determine a range of a corresponding target volume or organ-at-risk region through a coordinate value set.

The treatment plan information refers to data for guiding a radiation device to perform a complete radiation therapy course on the patient.

The treatment plan parameter refers to an execution parameter for controlling the radiation device to achieve a treatment objective. The treatment plan parameter may be a parameter for formulating a radiation therapy plan. A doctor or the processor may individually set the treatment plan parameter according to the treatment objective of the patient to ensure accuracy and efficacy of the therapy.

In some embodiments, the treatment plan parameter includes a dose allocation scheme, an irradiation direction and angle, a dose limit, or the like.

The dose allocation scheme is used to indicate a dose distribution for one or more radiation therapies in the radiation therapy plan. The dose allocation scheme includes how to distribute a radiation dose to the target volume and how to minimize a radiation dose to the organ-at-risk region. In some embodiments, the dose allocation scheme may include a spatial distribution and an intensity distribution of dose allocation.

The irradiation direction and angle are used to determine a direction, an angle, and an incident position of a radiation beam to ensure an optimal dose distribution and target volume coverage.

The dose limit (a dose threshold) is used to determine a radiation dose limit of the organ-at-risk region to ensure that excessive damage is not caused to the organ-at-risk region.

The treatment plan parameter may depend on a disease type, a treatment position, a severity of the disease of the patient, and availability of the radiation device. The present disclosure does not limit the treatment plan parameter.

In general, the doctors and a radiation therapist usually formulate a personalized treatment plan parameter based on clinical conditions and treatment guidelines to ensure that the patient obtains an optimal treatment outcome. For example, the doctor and the radiation therapist may manually formulate a treatment plan. As another example, the doctor and the radiation therapist may input a condition of the patient and a simulation CT image with delineated target volume and organ-at-risk region into a trained machine learning model to obtain treatment plan information including the treatment plan parameter.

Inputting the condition information of the patient and the simulation CT image with the delineated target volume and organ-at-risk region into a trained model to obtain the treatment plan information including the treatment plan parameter refers to a method of automatically obtaining a treatment plan using machine learning or artificial intelligence technology. Merely by way of example, a training process of the model includes:

Data preparation: preparing a training set. The training set includes, as samples, the condition information (medical record information of the patient) and the simulation CT image (with the delineated target volume and organ-at-risk region).

Model training: constructing a model to be trained using a machine learning or deep learning algorithm. The model to be trained takes, as input, the condition information and the simulation CT image as samples, and then outputs the treatment plan parameter. During the training process, the model to be trained learns how to generate an effective treatment plan based on the condition and image data.

Model validation: after the training is completed, validating the model. For example, inputting new patient data into the model, and then checking whether the treatment plan generated by the model meets a clinical standard or a recommendation of the doctor.

Model optimization: optimizing and improving the model based on a result of the validation to ensure performance and accuracy of the model.

In one implementation, a manner of obtaining the simulation CT information includes: obtaining a simulation CT image of the patient using a computed tomography device, and obtaining the simulation CT information according to a delineation operation on the target volume and the organ-at-risk region on the simulation CT image.

Exemplary CT devices may include a spiral CT, a spectral CT, a cone-beam CT, or the like.

The delineation operation refers to an operation in which the doctor or the radiation therapist marks the target volume and the organ-at-risk region on the simulation CT image. For example, the doctor circles the organ-at-risk region on the simulation CT image.

Merely by way of example, on the simulation CT image, the doctor or the radiation therapist may perform the delineation operation to mark the target volume (a target tissue or a tumor that needs to receive treatment) and the organ-at-risk (an organ that needs to avoid excessive radiation) inside a body of the patient. The doctor may formulate the radiation therapy plan on the simulation CT image to ensure that an irradiation dose is accurately projected onto the target volume of the patient while minimizing damage to the organ-at-risk. The radiation therapy plan refers to an overall plan for implementing the radiation therapy based on the simulation CT image. Similarly, the doctor or the radiation therapist may use software to automatically perform the delineation operation on the simulation CT image.

In some embodiments, the patient undergoes the CT scanning before the radiation therapy to generate the simulation CT image of the patient. The simulation CT image may capture an internal anatomical structure of the patient including the target volume and surrounding tissues and the organ-at-risk region. In some embodiments, the processor may be configured to analyze and process the simulation CT image using computer software to obtain the region segmentation information of the target volume and the organ-at-risk region, and use the simulation CT image and the corresponding region segmentation information as the simulation CT information.

Merely by way of example, the processor uses medical image display and processing software to mark boundary contours of the target volume and the organ-at-risk region on the simulation CT image based on the simulation CT image on which the delineation operation is completed. The boundary contours of the target volume and the organ-at-risk region are digitized to generate the region segmentation information.

Thus, through the simulation CT information, the doctor can be assisted in accurately locating an anatomical structure and a target region (the target volume) of the patient. The simulation CT information provides key information for precisely planning radiation therapy.

In S2, in a current fractionation of radiation therapy corresponding to the treatment plan information, obtaining surface optical image information of an irradiated region of the patient by measuring Cherenkov radiation emitted by the patient due to particle irradiation.

The current fractionation of radiation therapy refers to a current radiation therapy fractionation being performed in a complete radiation therapy course. As described above, the complete radiation therapy course includes a plurality of independent radiation therapy fractions, i.e., dividing a total radiation dose of the complete course into a plurality of fractions for irradiation. One independent radiation therapy fraction may be referred to as a fractionation of irradiation.

The Cherenkov radiation refers to a visible light signal that is generated due to interaction between the particle irradiation and tissue when the patient receives the radiation therapy, and that is capable of propagating to a body surface and being detected by an optical measurement device.

Merely by way of example, the Cherenkov radiation may be measured by the optical measurement device.

The optical measurement device refers to a device configured to measure the Cherenkov radiation emitted by the patient due to the particle irradiation. In some embodiments, the optical measurement device includes a light detector. In some embodiments, the light detector may include a silicon pixel array detector for measuring emitted photons. The photons are generated by interaction between the particle irradiation and the tissue. A property and a position of the particle irradiation may be determined by analyzing the light signal. In some embodiments, the optical measurement device may further include photodetectors, photomultiplier tubes, optical fibers, or the like.

The irradiated region refers to a three-dimensional spatial volume irradiated by the particle irradiation on the patient at a certain moment. In some embodiments, the particle irradiation may completely cover the target volume by irradiating a plurality of irradiated regions.

The surface optical image information refers to information indicating a dose distribution of the irradiation region of the patient. For example, the surface optical image information may indicate that an irradiation dose at a region A of the patient is 62 Gray, and an irradiation dose at a region B of the patient is 12 Gray. The surface optical image information may be displayed in a form of a dose distribution map to show a dose distribution situation in different regions of the patient.

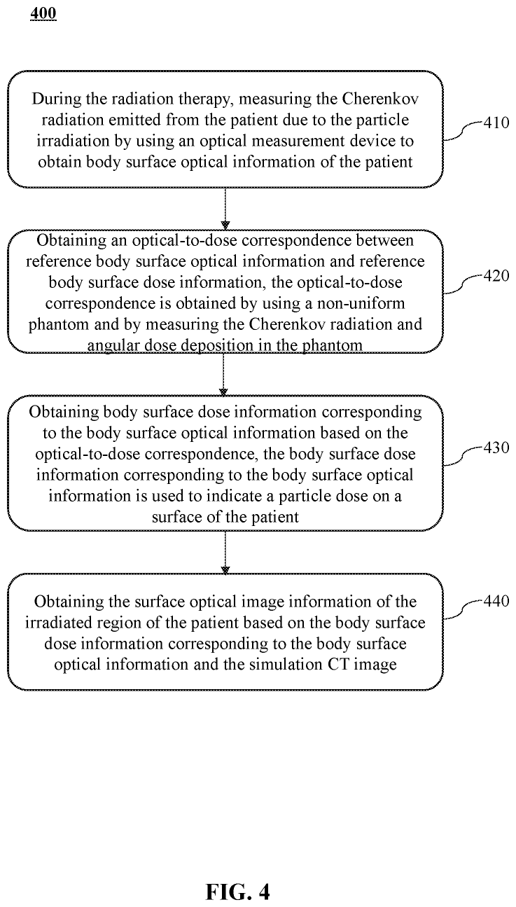

During a radiation therapy process corresponding to the treatment plan information, the optical measurement device may be used to measure the Cherenkov radiation emitted by the patient due to the particle irradiation, and real-time surface optical image information may be obtained based on the Cherenkov radiation to display a dose distribution of the irradiation region of the particle irradiation inside the body of the patient. More descriptions regarding a manner of obtaining the surface optical image information may be found elsewhere in the present disclosure (e.g., FIG. 4 and related descriptions thereof).

In some embodiments, before performing Step S2, the processor may perform positioning error detection on the patient. Step S2 is performed when a positioning of the patient meets a requirement. More descriptions regarding the positioning error detection may be found elsewhere in the present disclosure.

In S3, obtaining the dose distribution of the irradiated region based on the surface optical image information, the simulation CT information, and a preset mapping relationship, and obtaining irradiation dose information of the target volume and the organ-at-risk region based on the dose distribution of the irradiated region.

The preset mapping relationship refers to a conversion relationship between a secondary electron dose and a total dose received at any location inside the patient during the radiation therapy.

In one implementation, the preset mapping relationship is a correspondence between the secondary electron dose and the total dose obtained by simulating an interaction between a particle and the patient during treatment using a Monte Carlo method.

The total dose refers to a sum of energy deposition caused by all charged particles received at a point inside the patient.

The secondary electron dose refers to energy deposition caused by secondary electrons generated through interactions such as ionization and excitation. The Cherenkov radiation excited by the secondary electrons inside the patient may propagate to the body surface of the patient and be measured by the optical measurement device. A radiation intensity of the Cherenkov radiation is positively correlated with a spatial distribution of the secondary electron dose. Therefore, by measuring an intensity distribution of the Cherenkov radiation on the body surface of the patient and using an optical-to-dose correspondence, a secondary electron dose distribution on the body surface may be inversely derived, thereby obtaining the secondary electron dose inside the patient.

The Monte Carlo method is a numerical simulation technique based on random sampling for simulating an interaction between particles (for example, radiation rays, proton beams, or heavy ion beams) and patient tissue during treatment. Merely by way of example, the secondary electrons are generated by the interaction between the particles and the patient during treatment. Propagation and deposition processes of the secondary electrons in tissue may be simulated through the Monte Carlo method. Simultaneously, a distribution of the total dose, that is, total energy deposition caused by the particles in the patient tissue during treatment, is simulated using the Monte Carlo method. Thus, the secondary electron dose and a corresponding total dose at each point in the patient tissue may be obtained, thereby establishing a relationship between the secondary electron dose and the total dose (i.e., the preset mapping relationship).

An advantage of this approach is that using Monte Carlo simulation can accurately establish the relationship between the secondary electron dose and the total dose at each point in the patient tissue, thereby improving accuracy of a mapping between optical measurement and an actual dose.

The dose distribution refers to a deposition situation of radiation energy inside the patient, which may reflect a distribution of an absorbed dose inside the patient. The absorbed dose is radiation energy absorbed per unit mass of tissue.

The irradiation dose information refers to information used to indicate irradiation doses of different regions (including at least the target volume and the organ-at-risk region) in the radiation therapy. The irradiation dose information focuses more on the irradiation doses of the different regions. For example, the irradiation dose information indicates that an irradiation dose of a target volume A is 12 Gray, and indicates that an irradiation dose of an organ-at-risk region B is 2 Gray.

The irradiation dose refers to a radiation dose received by the target region (for example, the target volume) of the patient during the radiation therapy or radiation exposure, and may be expressed in units of Gray (Gy). The irradiation dose may represent a total amount of radiation to which the patient or a specific region is exposed during the radiation exposure, directly affecting efficacy of treatment and safety of the patient.

In some embodiments, the processor may determine body surface dose information according to the surface optical image information and the optical-to-dose correspondence. The secondary electron dose distribution inside the patient is calculated from the body surface dose information according to the simulation CT information. The dose distribution of the irradiated region is determined according to the secondary electron dose distribution via the preset mapping relationship. More descriptions regarding how to obtain the dose distribution of the irradiated region according to the surface optical image information, the simulation CT information, and the preset mapping relationship may be found elsewhere in the present disclosure (e.g., FIG. 4 and related descriptions thereof).

In some embodiments, the processor may use spatial contours of the target volume and the organ-at-risk region defined by the region segmentation information as an extraction mask to extract dose values located within contours of a plurality of regions from the dose distribution of the irradiated region. The extracted dose values are calculated to generate the irradiation dose information including, but not limited to, parameters such as an average dose, a maximum dose, or a dose-volume histogram for each region.

In S4, determining whether the treatment plan information for a subsequent fractionation of irradiation needs to be adjusted based on the irradiation dose information and the treatment plan information.

The processor may determine whether the treatment plan information of one or more subsequent radiation therapy fractions needs adjustment according to the irradiation dose information and the treatment plan information.

In one implementation, the treatment plan parameter includes a prescription dose and a tolerance dose limit.

Determining whether the treatment plan information for the subsequent irradiation needs adjustment according to the irradiation dose information and the treatment plan information includes:
determining whether the treatment plan information for the subsequent irradiation needs to be adjusted according to a difference between the irradiation dose information of the target volume and a prescription dose of the target volume required by the treatment plan information, and a difference between the irradiation dose information of the organ-at-risk region and a tolerance dose limit of the organ-at-risk region.

In some embodiments, the difference between the irradiation dose information (for example, the irradiation dose) of the target volume and the prescription dose of the target volume required by the treatment plan information may be referred to as a first difference, and the difference between the irradiation dose information (for example, the irradiation dose) of the organ-at-risk region and the tolerance dose limit of the organ-at-risk region may be referred to as a second difference.

The prescription dose refers to a total radiation dose planned to be received by the target volume. For example, for a prostate cancer lesion, the prescription dose is 78 Gy. The prescription dose may be set by the doctor based on experience.

The tolerance dose limit refers to a maximum radiation dose that the organ-at-risk region can safely withstand. For example, a tolerance dose limit of a spinal cord is 45 Gy to 50 Gy. The tolerance dose limit may be set by the doctor based on experience.

Merely by way of example, the processor may compare the irradiation dose of the irradiation dose information of the target volume with the prescription dose to determine the first difference, and compare the irradiation dose of the irradiation dose information of the organ-at-risk region with the tolerance dose limit to determine the second difference.

An advantage of this approach is that by monitoring the irradiation dose information and making adjustments based on specific conditions of an individual patient, the treatment plan information becomes more aligned with an actual situation, thereby improving precision of treatment.

In some embodiments, the processor may determine whether the first difference is less than a first difference threshold, and determine whether the second difference is less than a second difference threshold. When the first difference is less than the first difference threshold and the second difference is less than the second difference threshold, the treatment plan information for the subsequent irradiation fraction is determined not to need to be adjusted; otherwise, the treatment plan information needs to be adjusted. The first difference threshold and the second difference threshold may be processor defaults or set based on experience of the doctor. For example, the first difference threshold and the second difference threshold may be 5 Gray. For example, when the first difference is 0 and the second difference is 0, the treatment plan for the subsequent of irradiation does not need to be adjusted.

In S5, in response to determining that the treatment plan information for the subsequent fractionation of irradiation needs to be adjusted, adjusting the treatment plan information for the subsequent fractionation of irradiation based on the dose distribution of the irradiated region and a treatment prescription corresponding to the patient.

The treatment prescription refers to data carrying instructions for the radiation therapy formulated by the doctor. In some embodiments, the treatment prescription includes a parameter for the radiation therapy formulated by the doctor.

The parameter of radiation therapy may include a total radiation dose, a radiation dose per radiation therapy, and a radiation site. The total radiation dose refers to a total amount of radiation that a patient will receive (for different radiation sites) during an entire course of the radiation therapy. The total radiation dose is a cumulative value that considers the entire treatment cycle. The radiation dose per radiation therapy refers to a specified radiation dose (for the different radiation sites) for each individual radiation therapy. The radiation dose per radiation therapy may vary according to a condition of the patient, a type and a location of a tumor, and a progress of the treatment.

A process of adjusting treatment plan information may include re-planning a dose distribution for each region, adjusting a beam direction, or adjusting other treatment plan parameters to ensure the patient receives more precise treatment.

In some embodiments, the processor may generate reference treatment plan information according to the irradiation dose information and the treatment prescription corresponding to the patient. The processor may use the reference treatment plan information to update the treatment plan information of the patient.

In one implementation, the processor may input the irradiation dose information and the treatment prescription corresponding to the patient into a plan generation model to obtain the reference treatment plan information. The processor may use the reference treatment plan information to update the treatment plan information of the patient.

The plan generation model refers to a computer algorithm, a rule, or a machine learning model used to generate a reference treatment plan.

The reference treatment plan information refers to an optimized set of treatment plan data used to update the treatment plan information.

The plan generation model is trained to generate new treatment plan information according to the irradiation dose information and the treatment prescription. In some embodiments, the plan generation model may be the machine learning model, the artificial intelligence model, or another algorithm. The plan generation model may analyze existing irradiation dose information and the treatment prescription, and then generate the new treatment plan information. The new treatment plan information enables the dose distribution to be adjusted to better satisfy the treatment prescription.

An advantage of this approach is that personalized treatment plan information may be generated by using the irradiation dose information and the treatment prescription of each patient. The personalized treatment plan information helps ensure the treatment plan information matches a treatment need of the patient, thereby improving the effect of the treatment. If the irradiation dose information indicates that the dose distribution of the patient does not match the treatment prescription, the plan generation model may automatically adjust the treatment plan information to better satisfy a requirement of the doctor. The automatic adjustment helps reduce a dose error and improve the accuracy of the treatment. The present embodiment combines the irradiation dose information and the treatment prescription, and uses the plan generation model for plan adjustment. The embodiment thereby achieves personalized and efficient radiation therapy, reduces the need for manual intervention, and improves the efficiency of the treatment process.

Merely by way of example, the plan generation model is obtained by training a deep learning model. A training process of the plan generation model may include:

Data collection: a certain number of pieces of training data is collected. The training data includes a plurality of pieces of treatment plan information and corresponding particle doses on a surface of a phantom, surface optical image information of the phantom, etc. The training data is used to train the model so that the model may learn an association between different treatment plan information and dose distributions.

Data preparation: the collected training data is preprocessed to ensure a format and structure of the training data are suitable for training the model. For example, a preprocessing process may include standardization of data, normalization of data, and division of the data into a training set, a validation set, and a test set, etc.

Training implementation: the training data is input into a selected deep learning model, and parameters of the deep learning model are adjusted through a backpropagation algorithm. The training process includes a certain number of iterations until a preset training termination condition is satisfied.

Validation and evaluation: the validation set is used to monitor a performance of the trained plan generation model. The monitoring helps detect whether the model has problems such as overfitting or underfitting. The test set is used to evaluate the performance of the model to ensure a generalization ability of the model on new data.

Deployment and application: the trained plan generation model is applied to the radiation therapy mentioned in the present disclosure to generate personalized treatment plans according to specific conditions of patients.

By design, a preset deep learning model may be obtained by establishing an appropriate number of neuron computing nodes and a multi-layer operation hierarchy structure, and selecting suitable input and output layers. Through learning and tuning of the deep learning model, a functional relationship from input to output is established. The functional relationship can approximate a real association relationship as much as possible. The plan generation model thus trained may obtain corresponding reference treatment plan information based on the irradiation dose information and the treatment prescription corresponding to the patient. The plan generation model has a wide range of applications, and its output has high accuracy and high reliability.

The irradiation dose information and the treatment prescription corresponding to the patient are used as input. A deep learning-based plan generation model is used to process and analyze the input data. The plan generation model is trained to predict and generate the new reference treatment plan information. An advantage of this approach is that the deep learning-based plan generation model can quickly and accurately obtain the reference treatment plan information for updating the treatment plan information through the input data, thereby improving the efficiency and precision of data processing. The accuracy of the plan generation model may be improved through training and optimization of the deep learning model. For example, the processor may adjust a model parameter of the plan generation model according to personalized information of each patient to obtain a plan generation model suitable for each patient. The adjustment helps predict and generate the reference treatment plan information more accurately.

In some optional implementations, the plan generation model may be obtained by training. In other optional implementations, a pre-trained plan generation model may be adopted.

An advantage of this approach is that the treatment plan may be adjusted according to actual situations, achieving personalized radiation therapy and ensuring each patient receives treatment most suitable for the need of the patient. Acquiring the surface optical image information enables the doctor to monitor the treatment progress of the patient at any time to avoid unexpected situations and improve safety. By timely adjusting the treatment plan information, radiation damage to surrounding organ-at-risk regions may be minimized, thereby reducing side effects of the treatment. Meanwhile, by better irradiating the target volume, the treatment effect may be improved, thereby improving a tumor control rate.

In some embodiments of the present disclosure, the treatment plan information is timely adjusted by using a time interval between different irradiation fractions to ensure an optimal treatment effect. Meanwhile, a lifestyle, a physical condition, and a treatment response of the patient may change during the treatment process. A plurality of treatments allows the doctor to adjust the treatment plan information according to these changes in combination with adjustment suggestions provided by the present embodiment to better ensure a continuous effectiveness of the treatment.

In summary, compared with a simulation method using the water phantom, the method can accurately obtain the irradiation dose information for indicating the target volume and the organ-at-risk region of the patient by detecting the Cherenkov radiation. The method judges the quality of the treatment plan information by comparing acquired actual measurement data with theoretical calculation data. Clearly, the method provided by the embodiments of the present disclosure avoids a biological difference defect of the water phantom, considers individual differences of patients. This improves the accuracy of plan adjustment for radiation therapy and the safety of radiation therapy, and brings broader application prospects for tumor radiation therapy.

The above automated manner for obtaining the treatment plan parameter may serve as an auxiliary tool for the doctor. This helps improve the efficiency and quality of the radiation therapy, and adaptively adjusts the treatment plan for the subsequent fractionation of irradiation.

In some embodiments, before executing step S2, process 100 further includes: obtaining a detection result of the positioning error detection of the patient, and executing step S2 when the detection result indicates that positioning of the patient meets a preset positioning condition.

In medical radiation therapy, the positioning refers to a process of correctly positioning and placing the patient on the radiation device. A purpose of the positioning is to ensure that the radiation therapy precisely targets the target volume of the patient, to minimize damage to the organ-at-risk region, and to ensure that the treatment produces an optimal effect on a tumor or a disease.

The preset positioning condition refers to a pre-set standard for determining whether a position and a posture of the patient meet a precision required for treatment. For example, the preset positioning condition may be that a deviation between an actual body position of the patient and an ideal body position set in treatment plan information is within an acceptable range. The acceptable range refers to a tolerable deviation range. For example, the acceptable range may be a distance between an actual spatial position of a same part of the patient and an ideal spatial position set in the treatment plan information.

The positioning error detection refers to a process of detecting a deviation between the actual body position of the patient and the ideal body position before the radiation therapy.

Generally, before the radiation therapy, the patient undergoes a positioning process to position the patient to a treatment position. The detection result of the positioning error detection indicates a difference between the actual body position of the patient and the ideal body position. In response to a determination that the detection result indicates that the position of the patient is within the acceptable range, that is, the preset positioning condition is met, the radiation therapy continues.

The detection result of the positioning error detection may be represented by one or more of Chinese characters, letters, numbers, and symbols. For example, “qualified”, “Y”, “1”, “√”, or the like may be used to indicate that the positioning of the patient meets the preset positioning condition. “Unqualified”, “N”, “0”, “×”, or the like may be used to indicate that the positioning of the patient does not meet the preset positioning condition.

In some embodiments of the present disclosure, by executing the positioning error detection before the current fractionation of the radiation therapy corresponding to the treatment plan information, the actual body position of the patient can be ensured to match the ideal body position. This helps improve treatment precision, ensures that radiation accurately irradiates the target volume, and reduces the risk of mis-irradiating the organ-at-risk region.

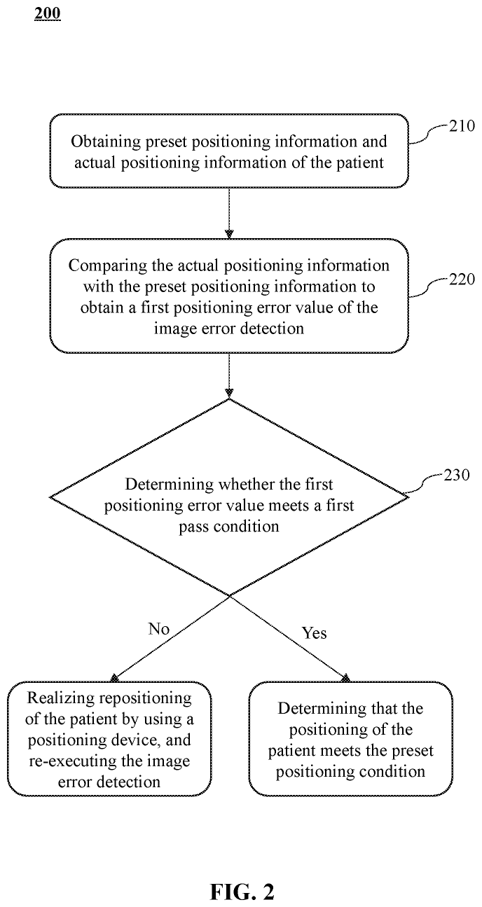

FIG. 2 is a flowchart of an exemplary process for image error detection according to some embodiments of the present disclosure

In some embodiments, the positioning error detection includes image error detection. As shown in FIG. 2, a process 200 includes the following steps:

In 210, obtaining preset positioning information and actual positioning information of the patient.

The preset positioning information refers to information reflecting an ideal position and an ideal posture that the patient should be in when receiving radiation therapy. For example, the preset positioning information may include a preset positioning image. The preset positioning information is usually determined in advance based on a treatment plan. The preset positioning information indicates the ideal position and the ideal posture that the patient should be in.

The actual positioning information refers to information about an actual position and an actual posture that the patient is in when receiving the radiation therapy. For example, the actual positioning information may include an actual positioning image. The actual positioning image may display the actual position and the actual posture that the patient is in.

In some embodiments, obtaining the actual positioning information includes:
obtaining the actual positioning information of the patient by using a radiographic imaging device.

Merely by way of example, the doctor positions the patient on a radiation device according to the preset positioning information. Then, the radiographic imaging device (e.g., an X-ray machine or other imaging device) is used to image the patient. The imaging process aims to capture the actual positioning information including the position and the posture of the patient. The radiographic imaging device is, for example, a device including an X-ray machine or computed tomography (CT scanning).

In 220, comparing the actual positioning information with the preset positioning information to obtain a first positioning error value of the image error detection.

The first positioning error value refers to a comparison result between the actual positioning information and the preset positioning information. For example, the first positioning error value may be a deviation between an image corresponding to the actual positioning information and an image corresponding to the preset positioning information.

Merely by way of example, the processor may perform a rigid body transformation comparison between the actual positioning information and the preset positioning information in a same coordinate system (e.g., a treatment room coordinate system, i.e., a three-dimensional Cartesian coordinate system of a space where the patient receives the radiation therapy). The processor may calculate deviation values of the actual positioning information relative to the preset positioning information in a plurality of degrees of freedom (e.g., translation deviations on an X-axis, a Y-axis, and a Z-axis, and rotation deviations about the X-axis, the Y-axis, and the Z-axis) are as the first positioning error value.

In 230, when the first positioning error value does not meet a first pass condition, realizing repositioning of the patient by using a positioning device, and re-executing the image error detection, and when the first positioning error value meets the first pass condition, determining that the positioning of the patient meets the preset positioning condition.

The first pass condition refers to a standard for judging whether the first positioning error value meets a requirement of the image error detection. For example, the first pass condition may be that the first positioning error value is within a tolerance range. The tolerance range refers to a range of allowable error or variation, which may be expressed as a percentage or a numerical value. For example, the first pass condition may be that a deviation value on the X-axis is less than 3 mm, a deviation value on the Y-axis is less than 2 mm, and a deviation value on the Z-axis is less than 4 mm. In response to a determination that the deviation values in the first positioning error value meet the above requirements, the processor may determine that the first positioning error value meets the first pass condition.

In this embodiment, the actual positioning information and the preset positioning information may be compared to calculating the first positioning error value, which indicates a difference between the actual position and the actual posture of the patient and an expected position and an expected posture in the treatment plan. A detection result of the image error detection is determined according to the first positioning error value and the first pass condition. In response to a determination that the first positioning error value meets the first pass condition, that is, the detection result of the image error detection indicates that the positioning of the patient meets the preset positioning condition, the treatment continues. In response to a determination that the first positioning error value does not meet the first pass condition, that is, the detection result of the image error detection indicates that the positioning of the patient does not meet the preset positioning condition, the position and the posture of the patient need to be adjusted to ensure treatment accuracy. For example, an adjustment manner may include using the positioning device such as a treatment couch to readjust the position of the patient to meet the preset positioning condition.

This embodiment ensures correct positioning of the patient during the radiation therapy process through the image error detection. An advantage of this is that the image error detection can ensure that the actual position of the patient is consistent with a target position in treatment plan information, which helps improve treatment precision, ensures that radiation can accurately irradiate a lesion region, and minimizes an impact on surrounding normal tissue.

In the image error detection process, by comparing the actual positioning information and the preset positioning information and calculating the first positioning error value, the patient can be ensured to be in a correct position and a correct posture during treatment, which helps improve treatment precision and ensures that radiation particles accurately irradiate a target region (a target volume).

In some embodiments, before the image error detection, the positioning error detection further includes laser error detection. The laser error detection is implemented based on a laser device. An exemplary laser device may include a laser positioning system, a laser scanner, or the like.

The laser error detection refers to a process of using a laser system to detect a difference between a position or a posture of a patient and an ideal position or an ideal posture. For example, a laser beam is emitted by the laser device. The laser beam forms one or more light spots on the patient. Then, a position of the patient or whether the posture of the patient is correct is determined by detecting a position of the one or more light spots.

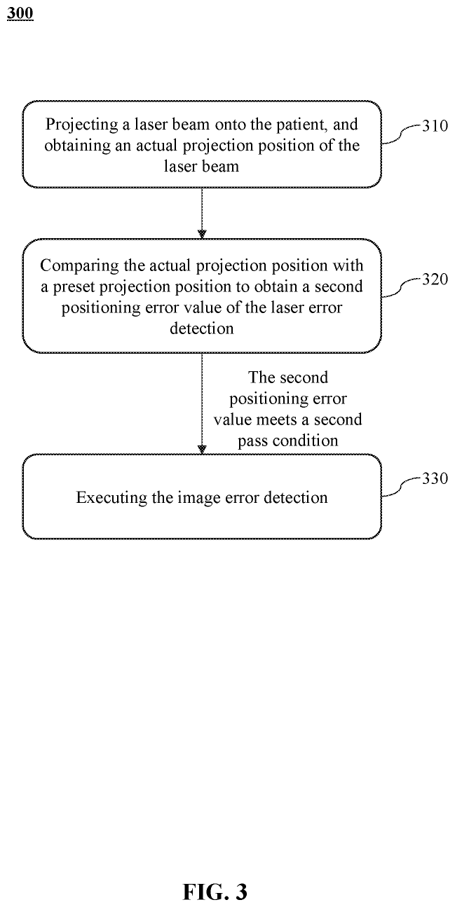

FIG. 3 is a flowchart of an exemplary process for the laser error detection according to some embodiments of the present disclosure

As shown in FIG. 3, a process 300 for the laser error detection includes the following steps:

In 310, projecting a laser beam onto the patient, and obtaining an actual projection position of the laser beam.

The actual projection position refers to an actual position of a visible light spot formed by the laser beam on the body surface of the patient. In some embodiments, the actual projection position may be visually observed by the doctor or the radiation therapist, or identified and measured by an optical sensor or a camera.

In 320, comparing the actual projection position with a preset projection position to obtain a second positioning error value of the laser error detection.

The preset projection position refers to an ideal position where the laser beam should be projected onto the body surface of the patient, which is preset.

Merely by way of example, the processor may read a coordinate parameter related to the laser error detection from treatment plan information. The coordinate parameter defines a target spatial position where the laser beam should be projected onto the body surface of the patient, i.e., the preset projection position.

The second positioning error value refers to a result of comparison between the actual projection position and the preset projection position. For example, the second positioning error value may be a distance between the actual projection position and the preset projection position in a treatment room coordinate system.

Merely by way of example, the processor may convert the preset projection position and the actual projection position into a same coordinate system (e.g., the treatment room coordinate system). The processor calculates a distance between the converted preset projection position and the actual projection position as the second positioning error value.

In 330, when the second positioning error value meets a second pass condition, executing image error detection.

The second pass condition refers to a standard for determining whether the second positioning error value meets a requirement of the laser error detection. For example, the second pass condition is that a distance between the actual projection position and the preset projection position in the treatment room coordinate system is less than a preset distance (e.g., 4 mm).

In some embodiments, when the second positioning error value meets the second pass condition, the processor receives pass information corresponding to the laser error detection and executes the image error detection. Merely by way of example, the pass information may be in a form of a digital signal, an indicator light, a specific display on a computer screen, or the like. The pass information reminds the doctor that a positioning of the patient has reached a required standard (e.g., the second pass condition), and a subsequent step, such as the image error detection, may be executed. The above steps help ensure an accurate position of the patient during treatment, thereby improving precision and safety of the treatment.

When the second positioning error value does not meet the second pass condition, the positioning of the patient needs to be adjusted, and then the laser error detection is executed again until the laser error detection is passed. For example, the processor may control a positioning device to adjust the positioning of the user based on the second positioning error value, or send the second positioning error value to a user device (see description below) to inform the doctor that the positioning of the patient needs to be adjusted.

An advantage of this approach is that the laser error detection helps ensure that an actual position of the patient on a treatment couch is consistent with an expected position. The laser error detection helps improve accuracy of the treatment and ensures that a radiation dose is precisely irradiated to a target region (a target volume). The laser error detection is a fast and non-invasive process and does not involve any component with radiation risk. Executing the image error detection after confirming that the position and posture of the patient are correct can ensure that the patient is in a suitable position before receiving the radiation therapy, reducing additional radiation exposure caused by the image detection. If the laser error detection has confirmed that the position and posture of the patient are substantially correct, interventions such as repositioning or adjusting the treatment couch are no longer required before executing the image error detection. This can reduce patient discomfort and anxiety during the treatment process and improve patient comfort.

In summary, using the laser error detection as a first step before treatment and executing the image error detection after the laser error detection can improve safety, comfort, and efficiency of the treatment while minimizing radiation risk to the patient. The above phased detection manner helps ensure that the patient is in an optimal state when receiving the radiation therapy.

In some embodiments, when the first positioning error value does not meet the first pass condition, realizing the repositioning of the patient by using the positioning device includes: generating a positioning adjustment strategy based on the first positioning error value, and controlling the positioning device to execute the positioning adjustment strategy to realize the repositioning of the patient.

The positioning adjustment strategy refers to a pose adjustment scheme for guiding the positioning device to reposition to eliminate deviation. The positioning adjustment strategy may be used to correct the position and the posture of the patient to adjust the position and the posture of the patient to a preset position and a preset posture. Taking the treatment couch as an example, the positioning adjustment strategy may include actions or operations such as lifting, rotating, or tilting of the couch.

In some embodiments, the processor may generate the positioning adjustment strategy through reverse equal compensation based on the first positioning error value.

In some embodiments, the positioning device includes the treatment couch, a robotic positioning system, or the like. For example, the treatment couch typically has a plurality of degrees of freedom and may control a position, a rotation, and a tilt of the patient. The treatment couch is configured to perform automatic or semi-automatic adjustment according to the positioning adjustment strategy to ensure that the patient is in a correct position and posture. As another example, the robotic positioning system may include a plurality of robotic hands or robotic arms for precisely controlling the position and the posture of the patient according to the positioning adjustment strategy.

In some embodiments, the robotic positioning system or the treatment couch may be integrated with a radiographic imaging device and a laser device to monitor and adjust the position of the patient.

In some embodiments of the present disclosure, by detecting and adjusting the position and the posture of the patient, it is ensured that the target volume receives an accurate radiation dose during treatment, and errors are minimized. By accurately adjusting the position and the posture of the patient, discomfort during the treatment process can be reduced, improving the treatment experience of the patient.

FIG. 4 is a flowchart of an exemplary process for acquiring surface optical image information according to some embodiments of the present disclosure.

As shown in FIG. 4, the process 400 includes following steps:

Step 410: During radiation therapy process, measure Cherenkov radiation emitted by the patient due to particle irradiation through an optical measurement device to obtain body surface optical information of the patient.

The body surface optical information refers to data reflecting a distribution of Cherenkov light intensity detected from a skin surface of the patient. For example, the body surface optical information may include a body surface optical image.

In 420, obtaining an optical-to-dose correspondence between reference body surface optical information and reference body surface dose information, the optical-to-dose correspondence is obtained by using a non-uniform phantom and by measuring the Cherenkov radiation and angular dose deposition in the phantom.

The reference body surface optical information refers to data reflecting a distribution of Cherenkov light intensity detected from a surface of the phantom. For example, the reference body surface optical information may include a reference body surface optical image.

The reference body surface dose information refers to actual dose distribution data at or near a body surface of the phantom. For example, the reference body surface dose information may be an actual dose distribution image at a position 1 mm from the body surface of the phantom. In some embodiments, the reference body surface dose information may be obtained by directly measuring with a micro-dosimeter implanted in the phantom, or by calculation through a Monte Carlo method.

The phantom refers to a physical entity model that simulates the radiation and light transmission characteristics of patient tissues. For example, the phantom may be a non-uniform simulated human tissue phantom.

The angular dose deposition refers to dose deposition at different angles caused by the interaction of radiation particles with atoms in a material as the radiation particles propagate through the material. Propagation and deposition of particle irradiation in tissues can more comprehensively consider by using information of the angular dose deposition.

The optical-to-dose correspondence refers to a relationship between the body surface optical information indicating intensity of the Cherenkov radiation and a particle dose on a surface of the patient, which is determined through a series of measurements or simulation analyses. For example, the optical-to-dose correspondence may be represented by a model, a function, a table, a matrix, a formula, etc., and the present disclosure does not limit this.

In some embodiments, the optical-to-dose correspondence is obtained by using the non-uniform simulated human tissue phantom and by measuring the Cherenkov radiation and the angular dose deposition in the phantom.

Merely by way of example, the doctor or the radiation therapist may use the non-uniform simulated human tissue phantom. In one or more particle irradiation experiments with a known dose, the doctor or the radiation therapist may use the optical measurement device to measure a distribution of Cherenkov light generated on the surface of the phantom, i.e., to obtain the reference body surface optical information. The doctor or the radiation therapist may use a dose detector pre-embedded inside the phantom to measure a true absorbed dose at a point, i.e., to obtain the reference body surface dose information. By analyzing a plurality of sets of experimental data, a quantitative conversion relationship between the reference body surface optical information and the reference body surface dose information may be established, i.e., the optical-to-dose correspondence.

In 430, obtaining body surface dose information corresponding to the body surface optical information based on the optical-to-dose correspondence. The body surface dose information corresponding to the body surface optical information is used to indicate the particle dose on the surface of the patient.

The body surface dose information refers to actual dose distribution data on or near a body surface of the patient. For example, the reference body surface dose information may be an actual dose distribution image at a position 1 mm from the body surface of the patient.

Merely by way of example, the optical-to-dose correspondence may be a lookup table reflecting a quantitative relationship between the reference body surface optical information and the reference body surface dose information. The processor may extract optical characteristic values (e.g., grayscale values) of a plurality of pixel points in the body surface optical information. The processor may use the optical characteristic values as lookup indices, and perform a matching query in the lookup table to obtain corresponding body surface dose information. The lookup table may be preset by the doctor.

In 440, obtaining the surface optical image information of the irradiated region of the patient based on the body surface dose information corresponding to the body surface optical information and a simulation CT image.

The processor may infer the body surface dose information of the patient based on the established optical-to-dose correspondence and an output of the optical measurement device. The processor may combine the body surface dose information and the simulation CT image of the patient to calculate the surface optical image information of the irradiated region of the patient.

Merely by way of example, the processor may be configured to extract a three-dimensional surface model of the body surface of the patient from the simulation CT image. The processor may establish a spatial coordinate transformation relationship between a two-dimensional image plane where the body surface dose information is located and the three-dimensional body surface model, according to a known spatial position of the optical measurement device. According to the spatial coordinate transformation relationship, the processor may map particle doses of the plurality of pixel points in the body surface dose information to corresponding three-dimensional spatial points on the three-dimensional body surface model, to generate a data set corresponding to the particle doses and the three-dimensional body surface model, i.e., the surface optical image information of the irradiated region of the patient

An advantage of this approach is that dose distribution of the patient can be monitored in real time during treatment to ensure the particle doses are delivered as planned, thereby improving treatment precision and safety. Combining optical measurement and image processing techniques to provide key information about the dose distribution of the patient (the surface optical image information of the irradiated region of the patient) in the radiation therapy helps improve treatment safety and efficacy.

In some embodiments of the present disclosure, using the phantom to simulate an actual human condition helps establish a more realistic and accurate optical-to-dose correspondence. This is because characteristics of human tissues, including parameters such as tissue density, vary among different individuals. Using the non-uniform simulated human tissue phantom can better account for individual differences, making the established optical-to-dose correspondence more universal.

In summary, in some embodiments of the present disclosure, the established optical-to-dose correspondence is more accurate, thereby enabling better mapping of optical information to the body surface dose information in practical applications.

In some embodiments, during the radiation therapy, the method further includes: comparing irradiation dose information with a dose threshold for a current fractionation of the radiation therapy. When an irradiation dose of an organ-at-risk region exceeds a corresponding dose threshold thereof, abnormal prompt information is generated and sent to a user device.

The dose threshold refers to a maximum irradiation dose that a target volume and the organ-at-risk region can receive during the current fractionation of the radiation therapy. For example, the dose threshold is 60 Gray, 50 Gray, 40 Gray, 34 Gray, 33 Gray, 20 Gray, 16 Gray, 12 Gray, or 4 Gray.

Merely by way of example, if a tolerance dose limit for a spinal cord is 45 Gy, and treatment is divided into 30 fractions with an average of 1.5 Gy per fraction, then the dose threshold for the current fractionation of the radiation therapy may be set to 1.0 Gy.

It should be noted that there are two situations: a situation where the known irradiation dose of irradiation dose information exceeds the dose threshold for the current fractionation of the radiation therapy and a situation where the known irradiation dose of the irradiation dose information does not exceed the dose threshold for the current fractionation of the radiation therapy.

The abnormal prompt information refers to information used to alert the doctor that the irradiation dose of the organ-at-risk region exceeds a standard in the current fractionation of the radiation therapy. For example, the abnormal prompt information may include detailed information about dose exceeding the standard, which is used to guide the doctor to adjust treatment plan information to reduce risk, or to suggest that the doctor take measures to reduce the risk.

In this embodiment, during the radiation therapy, the processor may obtain the surface optical image information and compare the surface optical image information with a preset dose threshold to monitor whether an actual irradiation dose of the organ-at-risk region during treatment is within a reasonable range. When the irradiation dose of the organ-at-risk region exceeds the preset dose threshold during the comparison, the processor may generate the abnormal prompt information.

In some embodiments, the processor may send the abnormal prompt information to the user device of the doctor. For example, the user device may be a computer, a mobile phone, or a tablet computer. The doctor may adjust treatment plan information according to the abnormal prompt information to ensure patient safety and treatment efficacy.

An advantage of this approach is that it allows real-time monitoring of the dose distribution of the patient during the radiation therapy to pay attention to the irradiation dose of the organ-at-risk region, which helps timely identify potential treatment issues. By comparing the actual irradiation dose with the dose threshold, a situation where dose exceeds the standard may be detected early, thereby minimizing harm to an organ-at-risk of the patient and improving treatment safety. Timely abnormal prompt information can prompt the doctor to take measures and adjust the treatment plan information to ensure treatment meets expected quality standards and improve treatment effectiveness.

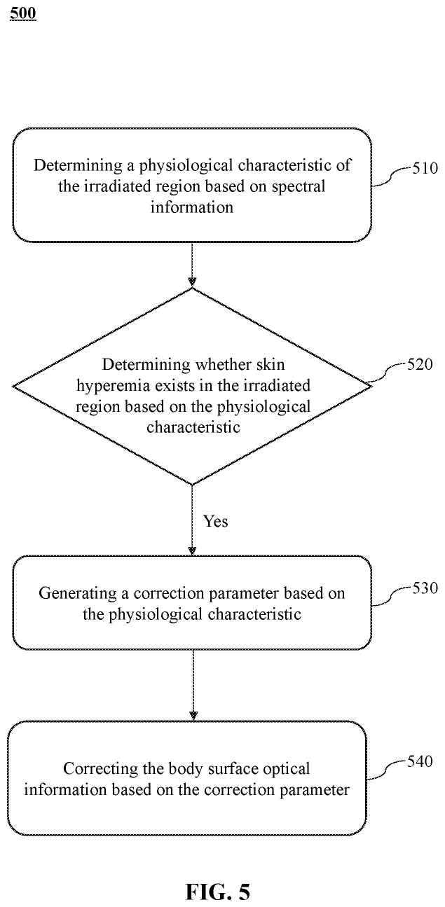

FIG. 5 is a flowchart of an exemplary process for correcting body surface optical information according to some embodiments of the present disclosure

In some embodiments, a process 500 may be executed based on the processor. As shown in FIG. 5, the process 500 includes the following steps:

In 510, determining a physiological characteristic of an irradiated region based on spectral information.

The spectral information refers to optical data containing parameters of wavelength (color) and intensity. For example, the spectral information may be a curve of light intensity varying with wavelength. In some embodiments, the spectral information may include a residual light intensity of light (e.g., Cherenkov radiation, ambient light, etc.) that passes through skin, is absorbed by blood, and is reflected/emitted back. In some embodiments, the spectral information may be obtained by a hyperspectral camera.

The physiological characteristic refers to a physiological parameter of a tissue in the irradiated region. For example, the physiological characteristic may include a total blood volume and an oxygen saturation of a plurality of pixel points in the body surface optical information (e.g., a two-dimensional light intensity distribution image) of the irradiated region.

The total blood volume refers to a filling degree of subcutaneous blood vessels in the irradiated region. The oxygen saturation refers to a metabolic state or a hypoxic condition of the tissue in the irradiated region.

In some embodiments, for a plurality of pixel points in the spectral information, the processor is configured to, for each of a plurality of wavebands, use the Beer-Lambert law to establish a calculation formula (1) for a total absorption amount of light by blood and the light intensity:

$$A ( λ ) \approx [ {ε}_{HbO 2} ( λ ) \cdot {C}_{HbO 2} + {ε}_{Hb} ( λ ) \cdot {C}_{Hb} ] \cdot L \qquad ( 1 )$$

A(λ) denotes an absorbance at a wavelength λ (calculated from a light intensity measured by the hyperspectral camera). ε_{HbO2} and ε_{Hb} denote molar extinction coefficients of two types of hemoglobin, which are known physical constants. C_{HbO2} and C_{Hb} denote concentrations of oxyhemoglobin (HbO2) and deoxyhemoglobin (Hb), respectively. L denotes an optical pathlength (a distance that light travels in the tissue), which may be set based on experience.

In practical application, for spectral information acquired by the hyperspectral camera and containing M× N pixel points, the absorbance A, the concentrations C_{HbO2} and C_{Hb}, and the optical pathlength L are all defined as values at each pixel point (i, j). That is, the processor independently calculates a set of physiological characteristic parameters for each pixel point in the spectral information.

The hyperspectral camera executes measurements at q different central wavelengths (wavebands) λ₁, λ₂, . . . , λ_{q}. Therefore, the absorbance A and the molar extinction coefficients ε_{HbO2} and ε_{Hb} are all related to a specific waveband q. A relationship between the pixel point (i, j) and the waveband q follows a formula (2) below:

$$A ( i , j , {λ}_{q} ) \approx [ {ε}_{HbO 2} ( {λ}_{q} ) \cdot {C}_{HbO 2} ( i , j ) + {ε}_{Hb} ( {λ}_{q} ) \cdot {C}_{Hb} ( i , j ) ] \cdot L ( i , j ) \qquad ( 2 )$$

For a same pixel point (i, j), substituting absorbances λ(i, j, λ₁) . . . A(i, j, λ_{q}) measured at q different wavebands (q≥2) into the above formula (2) yields a system of equations containing q equations. The system of equations takes two concentration values C_{HbO2}(i, j) and C_{Hb}(i, j) of the pixel point as unknowns. By solving this overdetermined system of equations (e.g., using a least squares method), concentrations of oxyhemoglobin and deoxyhemoglobin for the pixel point may be obtained.

In some embodiments, the processor is configured to, according to the calculation formula (1) and parameters related to the light intensity (e.g., the absorbance, the molar extinction coefficients, the optical pathlength), solve to obtain the concentration of oxyhemoglobin and the concentration of deoxyhemoglobin (i.e., C_{HbO2} and C_{Hb}). According to the concentration of oxyhemoglobin and the concentration of deoxyhemoglobin, the oxygen saturation (StO₂) and the total blood volume (Cₜₒₜₐₗ) are obtained through formulas (3) and (4) below:

$${StO}_{2} = \frac{{c}_{HbO 2}}{{c}_{HbO 2} + {c}_{Hb}} \qquad ( 3 )$$

$${C}_{total} = {C}_{HbO 2} + {C}_{Hb} \qquad ( 4 )$$

It should be noted that the total absorption amount of the light by the blood equals the absorption amount by oxyhemoglobin plus an absorption amount by deoxyhemoglobin. Therefore, formula (1) adds the absorption amount by oxyhemoglobin and the absorption amount by deoxyhemoglobin to obtain the total absorption amount of the light by the blood.

It should be noted that the hyperspectral camera and a camera for acquiring the body surface optical information are usually coaxial or strictly calibrated. A plurality of pixel points of a hyperspectral image (e.g., the spectral information) and the plurality of pixel points of the body surface optical information correspond one-to-one in spatial position. The processor may align spatial positions of the plurality of pixel points of the body surface optical information of the irradiated region with the plurality of pixel points of the spectral information. The processor may determine physiological characteristics of the plurality of pixel points of the body surface optical information of the irradiated region according to physiological characteristics of the plurality of pixel points in the spectral information.

In 520, determining whether skin hyperemia exists in the irradiated region based on the physiological characteristic.

In some embodiments, the processor may determine whether an average total blood volume of the plurality of pixel points of the irradiated region is greater than a preset total blood volume threshold. In response to a determination that the average total blood volume is greater than the preset total blood volume threshold, the processor determines that the skin hyperemia exists in the irradiated region. The preset total blood volume threshold may be set by the doctor based on experience.

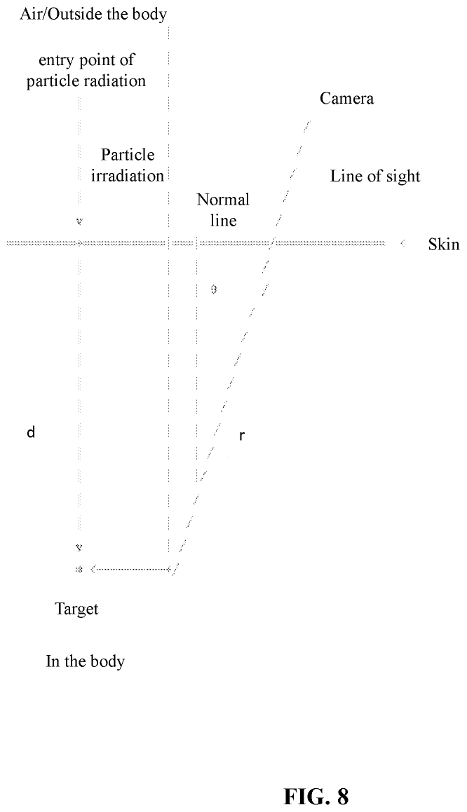

More descriptions regarding step 520 may be found elsewhere in the present disclosure (e.g., FIG. 8 and related descriptions thereof).

In 530, in response to determining that the skin hyperemia exists in the irradiated region, generating a correction parameter based on the physiological characteristic.

The correction parameter refers to a parameter used to compensate for attenuation or enhancement of an optical signal during transmission in the tissue. In some embodiments, a numerical range of the correction parameter is 0 to 5. For example, when light supplement correction is required, a value range of the correction parameter is 1 to 5. When light reduction correction is required, the value range of the correction parameter is 0 to 1.

In some embodiments, the processor may generate the correction parameter based on the Beer-Lambert law according to the physiological characteristic. The Beer-Lambert law describes an attenuation law of light after passing through an absorbing medium, as shown in formula (5) below:

$${I}_{out} = {I}_{in} \cdot {e}^{- {μ}_{a} \cdot L} \qquad ( 5 )$$

Iᵢₙ denotes a generated light intensity. Iₒᵤₜ denotes a light intensity measured by the hyperspectral camera. μₐ denotes an absorption coefficient. L denotes the optical pathlength. A relationship between the correction parameter K and Iᵢₙ, Iₒᵤₜ is expressed by a formula (6) below:

$${I}_{in} \approx {I}_{out} \cdot K \qquad ( 6 )$$

The following formula (7) is obtained from formulas (5) and (6):

$$K = {e}^{+ {μ}_{a} \cdot L} \qquad ( 7 )$$

When the exponent in formula (7) is positive, the exponent indicates that the light supplement correction is required.

Merely by way of example, the processor may determine an absorption characteristic of blood, and determine a mixed molar extinction coefficient of the blood for light of a specific wavelength according to the following formula (8):

$${ε}_{mix} ( λ ) = {StO}_{2} \cdot {ε}_{HbO 2} ( λ ) + ( 1 - {StO}_{2} ) \cdot {ε}_{Hb} ( λ ) \qquad ( 8 )$$

StO₂ denotes the blood oxygen saturation (for example, StO₂ may be 0.9 or 90%). ε_{HbO2} and ε_{Hb} are known physical constants (obtainable by looking up a table). A denotes a central wavelength at which the hyperspectral camera operates (for example, λ is 570 nm).

The processor may determine a concentration change of the blood according to the following formula (9):

$$Δ C = {C}_{measured} - {C}_{baseline} \qquad ( 9 )$$

C_{measured} denotes the total blood volume determined by formula (4). C_{baseline} denotes a baseline total blood volume measured before treatment. ΔC denotes a concentration change of the blood. If ΔC is a positive number, the positive number indicates that the skin hyperemia exists in the irradiated region (the light supplement correction is required, i.e., K is greater than 1). If ΔC is a negative number, the negative number indicates that skin ischemia exists in the irradiated region (the light reduction correction is required, i.e., K is less than 1).

The processor may generate the correction parameter by using the Beer-Lambert law according to the following formula (10):

$$K = exp ( ln ( 10 ) \cdot {ε}_{mix} ( λ ) \cdot Δ C \cdot L ) \qquad ( 10 )$$

The baseline total blood volume C_{baseline} and the optical pathlength L may be preset values.

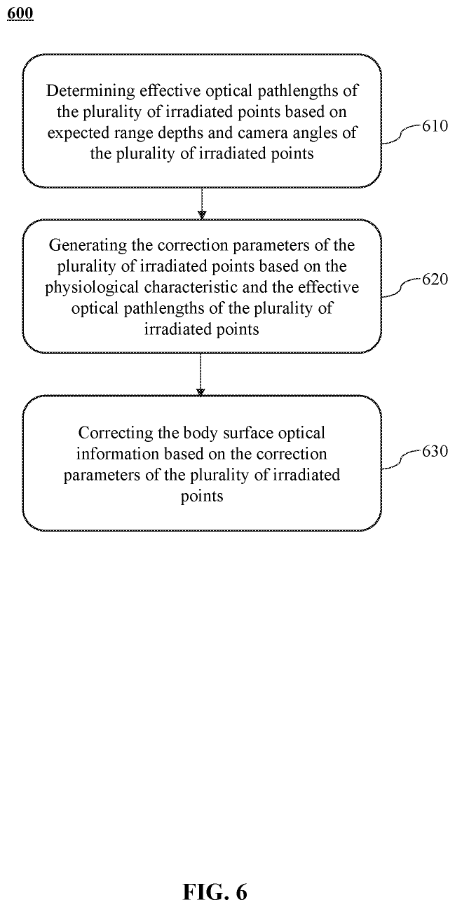

More descriptions regarding generating the correction parameter may be found elsewhere in the present disclosure (e.g., FIG. 6 and related descriptions thereof).

In 540, correcting the body surface optical information based on the correction parameter.

In some embodiments, the processor may correct the body surface optical information by multiplying light intensities Iₒᵤₜ of the plurality of pixel points in the body surface optical information by the correction parameter.

In some embodiments of the present disclosure, a false positive error in dose monitoring caused by factors such as the skin hyperemia can be eliminated by monitoring and compensating for optical signal attenuation caused by changes in physiological characteristics.

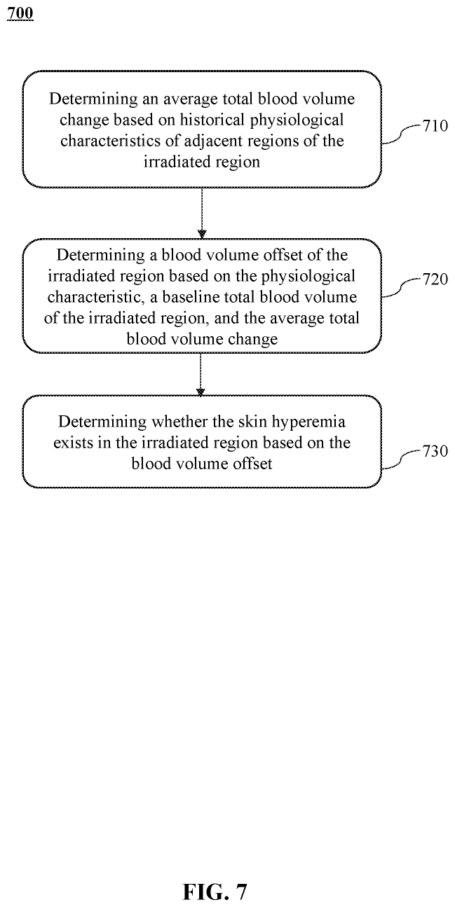

FIG. 6 is a flowchart of an exemplary process for generating the correction parameter according to some embodiments of the present disclosure. FIG. 7 is a schematic diagram of a camera angle according to some embodiments of the present disclosure.

In some embodiments, an irradiated region includes a plurality of irradiated points, and the plurality of irradiated points correspond to different correction parameters. In some embodiments, a process 600 may be performed by the processor. As shown in FIG. 6, the process 600 includes the following steps:

In 610, determining effective optical pathlengths of the plurality of irradiated points based on expected range depths and camera angles of the plurality of irradiated points.

The irradiated point refers to a point in the irradiated region that corresponds to a pixel point of body surface optical information of the irradiated region. In some embodiments, the plurality of irradiated points of the irradiated region may correspond one-to-one to a plurality of pixel points of the body surface optical information of the irradiated region.

The expected range depth refers to a depth at which particle irradiation (e.g., protons or heavy ions) is expected to penetrate tissue at a specific energy. In some embodiments, the processor may determine the expected range depths of the plurality of irradiated points based on a treatment plan (e.g., treatment plan information). As shown in FIG. 7, d represents the expected range depth.

The camera angle refers to an angle between a normal line perpendicular to a skin surface and a line connecting a camera (e.g., a hyperspectral camera) to the irradiated point. As shown in FIG. 7, θ represents the camera angle.

The effective optical pathlength r refers to an actual path length traveled by Cherenkov photons from a generation location to the skin surface where the Cherenkov photons are captured by the camera. As shown in FIG. 7, r represents the effective optical pathlength. Merely by way of example, the processor may determine the effective optical pathlength r based on the expected range depth d and the camera angle θ according to the following formula (11):

$$r = ( d / cos θ ) \cdot p \qquad ( 11 )$$

p denotes an empirical coefficient that considers a scattering characteristic of the tissue, and a value range of the empirical coefficient is 1.0 to 2.0.

In 620, generating the correction parameters of the plurality of irradiated points based on a physiological characteristic and the effective optical pathlengths of the plurality of irradiated points.

Merely by way of example, for each irradiated point, the processor may generate the correction parameter by replacing the optical pathlength L in the aforementioned formula (10) with the effective optical pathlength r.

In 630, correcting the body surface optical information based on the correction parameters of the plurality of irradiated points.

In some embodiments, the processor may correct the body surface optical information by multiplying light intensities Iₒᵤₜ of pixel points corresponding to the plurality of irradiated points by corresponding correction parameters K.

In some embodiments of the present disclosure, by matching the effective optical pathlengths with the actual range depths of the particle irradiation, a problem of inaccurate calculation of optical attenuation caused by variations in a depth of a radiation source is solved, thereby achieving precise optical correction in three dimensions.

FIG. 8 is a flowchart of an exemplary process for determining whether skin hyperemia exists according to some embodiments of the present disclosure.

In some embodiments, a process 800 may be performed by the processor. As shown in FIG. 8, the process 800 includes the following steps:

In 810, determining an average total blood volume change based on historical physiological characteristics of adjacent regions of an irradiated region.

The adjacent region refers to a region that is outside an irradiation field of radiation therapy and is not irradiated by particle irradiation within a field of view of a hyperspectral camera. For example, when the irradiated region is a heart, a lung is the adjacent region.

The historical physiological characteristics refers to physiological characteristics at a plurality of historical moments within a past period.

The average total blood volume change refers to an average fluctuation amplitude of a total blood volume measured in the adjacent region over time. The average total blood volume change may represent a systemic physiological baseline drift.

In some embodiments, the processor may calculate a mean of the physiological characteristics at the plurality of historical moments (e.g., total blood volumes at the plurality of historical moments) and use the mean as a first mean. The processor may determine a plurality of differences between the physiological characteristics at the plurality of historical moments and the first mean, and calculate a mean of the plurality of differences as a second mean. The processor may use the second mean as the average total blood volume change.

In 820, determining a blood volume offset of the irradiated region based on the physiological characteristic, a baseline total blood volume of the irradiated region, and the average total blood volume change.

The baseline total blood volume refers to a baseline total blood volume level of the patient measured before the radiation therapy.

Merely by way of example, the processor may obtain baseline spectral information after patient positioning is completed and before a first irradiation fraction. The processor may determine baseline physiological characteristics of the irradiated region based on the baseline spectral information. Based on the baseline physiological characteristics, the processor may determine a mean of total blood volumes of a plurality of pixels in baseline body surface optical information (e.g., a two-dimensional light intensity distribution image) of the irradiated region, and use the mean as the baseline total blood volume.

The blood volume offset refers to a net local blood volume change in the irradiated region caused solely by radiation after systemic changes are removed.

In some embodiments, the processor may determine the blood volume offset based on the physiological characteristics, the baseline total blood volume, and the average total blood volume change: the blood volume offset equals an average total blood volume of a plurality of pixels in body surface optical information of the irradiated region minus a sum of a baseline total blood volume and an average total blood volume change of the plurality of pixels.

In 830, determining whether the skin hyperemia exists in the irradiated region based on the blood volume offset.

In some embodiments, if the blood volume offset is greater than a preset blood volume offset threshold, the processor may determine that the skin hyperemia exists in the irradiated region. The preset blood volume offset threshold may be set by the doctor based on experience.

In some embodiments of the present disclosure, by introducing the adjacent region (i.e., a non-irradiated region) as a reference to remove the physiological baseline drift caused by systemic factors such as body temperature and emotion of the patient, thereby ensuring correction is executed only for radiation-induced local hyperemia.

In some embodiments, the processor may generate a correction parameter based on the blood volume offset and the physiological characteristic. For example, the processor may substitute the blood volume offset calculated in step 820 for ΔC in the aforementioned formula (10) to obtain a correction parameter K.

In some embodiments of the present disclosure, by using the blood volume offset, from which the systemic baseline drift has been removed to calculate the correction parameter, the specificity and signal-to-noise ratio of optical-to-dose correction are significantly improved.

In some embodiments, before determining whether treatment plan information for a subsequent fractionation of irradiation needs to be adjusted based on irradiation dose information and treatment plan information, the method further includes: determining a target irradiation dose of each of a plurality of future points based on an actual irradiation dose and a standard irradiation dose of each of a plurality of historical points in an irradiated region. The method further includes: generating a dose adjustment instruction based on the target irradiation dose of each of the plurality of future points, and sending the dose adjustment instruction to a beam controller. The dose adjustment instruction is configured to control the beam controller to adjust a dwell time of a particle beam pulse at each of the plurality of future points.

A point refers to a discrete irradiation dwell point preset on a movement trajectory of particle irradiation when a radiation device executes radiation therapy on the irradiated region. For example, 100 points are preset on the movement trajectory of the particle irradiation, and the particle irradiation irradiates sequentially from point 1, point 2, . . . , to point 100.

The historical position point refers to a point that has already been irradiated by the particle irradiation within a historical period. For example, when the particle irradiation is irradiating point 50, points 1 to 49 may be historical position points. The historical period is a period preceding a current moment, and a duration of the historical period is set based on experience.

The actual irradiation dose refers to an actual dose value received by a point from irradiation by the radiation device. For example, an actual irradiation dose of the point 50 is 14 Gray.

The standard irradiation dose refers to a pre-planned theoretical radiation dose value that should be achieved at a specific point. For example, a standard irradiation dose of the point 50 is 20 Gray. Merely by way of example, standard irradiation doses of the plurality of points may be different. The standard irradiation doses of the plurality of position points may be determined via a treatment plan (e.g., the treatment plan information).

The future point refers to a point that is to be irradiated by the particle irradiation within a future period. For example, when the particle irradiation is irradiating the point 50, points 51 to 100 may be future position points. The future period is a period following the current moment, and a duration of the future period is set based on experience.

The target irradiation dose refers to a dose that a next point needs to receive from irradiation by the radiation device. For example, a standard irradiation dose for the point 1 in the treatment plan information is 10 Gy, an actual irradiation dose for the point 1 is 8 Gy, a deviation is −2 Gy, and a target irradiation dose for point 2 is 12 Gy.

Merely by way of example, the target irradiation dose may be determined via a first vector database. The processor may construct the first vector database based on historical data. The first vector database includes a plurality of feature vectors and labels thereof. The processor may use historical actual irradiation doses and historical standard irradiation doses at the plurality of historical points as feature vectors. The processor may determine a label in the following manner. The processor may determine a plurality of historical radiation therapy records corresponding to a feature vector. The processor may extract a set of historical irradiation doses executed after a moment corresponding to the feature vector from the plurality of historical radiation therapy records. The processor may calculate a deviation between actual irradiation doses and standard irradiation doses after execution of a set of historical irradiation doses in the plurality of historical radiation therapy records. The processor may define a historical irradiation dose executed in a historical radiation therapy record with a smallest deviation among the plurality of historical radiation therapy records as the label for the feature vector.

The processor may generate a target vector based on actual irradiation doses and standard irradiation doses at the plurality of historical points in a current fractionation of the radiation therapy. The processor may obtain a feature vector with a highest similarity to the target vector from the first vector database via vector similarity matching, and use a label corresponding to the feature vector as a target irradiation dose corresponding to the target vector.

The dose adjustment instruction refers to a control instruction generated by the processor and sent to the beam controller. For example, the dose adjustment instruction includes a dwell time of the particle irradiation at the next point (i.e., a next point to be irradiated by the particle irradiation within the future period).

The dwell time refers to an irradiation duration of the particle radiation at the plurality of points along its movement trajectory. For example, the particle irradiation irradiates the point 50 for 2 seconds.

The beam controller refers to a core control subsystem in the radiation device (e.g., a proton or heavy ion particle therapy device). The beam controller is configured to control the dwell time of the particle irradiation.

In some embodiments, the processor may determine the dwell time required to achieve the target irradiation dose through a first preset table. The first preset table includes a correspondence between the target irradiation dose and the dwell time. The first preset table may be set by the doctor based on experience.

In some embodiments of the present disclosure, by adjusting dwell times of subsequent points through real-time feedback, dose deviations during a treatment process can be dynamically compensated, thereby improving the accuracy of a dose distribution of the irradiated region. Furthermore, compared to adjusting other parameters of the beam controller (e.g., adjusting a beam intensity of the particle irradiation), adjusting the dwell time is a manner with relatively high control precision and relatively low cost. For example, adjusting the other parameters requires adjusting an ion source or an extraction magnet. Such an operation typically requires a long time, and the beam intensity of the particle irradiation fluctuates during the adjustment process, making it difficult to achieve a required irradiation dose.

It should be noted that, during a radiation therapy process, two situations are known: the irradiated region is not completely scanned and the irradiated region is completely scanned. Scanning refers to irradiating the irradiated region using the particle irradiation.

In some embodiments, in response to determining that the irradiated region is completely scanned, the processor may generate a rescanning instruction including a rescanning path and a rescanning dwell time through a path generation model based on a shape feature of the irradiated region, the actual irradiation dose of each of the plurality of historical points, and the standard irradiation dose. The rescanning instruction is configured to control the radiation device to rescan the irradiated region based on the rescanning path, and to stay for the rescanning dwell time at each of a plurality of points on the rescanning path.

The shape feature refers to a size and a shape of the irradiated region. In some embodiments, the size of the irradiated region includes an internal spatial volume of the irradiated region, a body surface area of the irradiated region, or the like.

The rescanning instruction refers to a control instruction generated by the processor and sent to the radiation device (e.g., the beam controller of the radiation device). The rescanning instruction is configured to guide the radiation device to execute a supplementary scan.

The rescanning path refers to a movement trajectory of the particle irradiation in the supplementary scan executed to compensate for an insufficient dose after the irradiated region is completely scanned.

The rescanning dwell time refers to an irradiation duration of the particle irradiation at the plurality of points on the rescanning path.

In some embodiments, the processor may compare a mean of the actual irradiation doses of the plurality of points within the irradiated region with a mean of the standard irradiation doses. In response to the mean of the actual irradiation doses of the plurality of points within the irradiated region being less than the mean of the standard irradiation doses, the processor generates the rescanning instruction through the path generation model.

The path generation model is a model configured to generate the rescanning instruction. In some embodiments, the path generation model may be a machine learning model. The machine learning model may include, but is not limited to, a Deep Neural Network (DNN) model, a Graph Neural Network (GNN) model, a support vector machine model, a k-nearest neighbor model, a decision tree model, or a combination of one or more thereof.

In some embodiments, an input of the path generation model includes the shape feature of the irradiated region, the actual irradiation dose of each of the plurality of historical points, and the standard irradiation dose. An output of the path generation model includes the rescanning instruction.

In some embodiments, the path generation model may be the DNN model. The DNN model may be obtained through training based on at least one set of first training samples and corresponding first labels. A first training sample includes historical actual irradiation doses and historical standard irradiation doses of a plurality of historical position points of a plurality of historical irradiated regions. The first label includes the historical rescanning instruction that yields the smallest difference between the historical actual irradiation dose and the historical standard irradiation dose of the irradiated region after rescanning, among a plurality of historical rescanning instructions corresponding to the first training sample.

During training, the first training sample is input into an initial path generation model, and a loss function is constructed based on an output of the initial path generation model and the first label. Parameters of the initial path generation model are iteratively updated based on the loss function until a preset training condition is satisfied. When the preset training condition is satisfied, the training ends, and a trained path generation model is obtained as the path generation model. The preset training condition may include, but is not limited to, convergence of the loss function, a training epoch reaching a threshold, or the like.

In some embodiments of the present disclosure, by performing an average dose assessment after scanning of the irradiated region is completed and executing a supplementary scan, a total dose ultimately received by the irradiated region is ensured to meet treatment plan requirements, preventing the insufficient dose from affecting therapeutic efficacy.

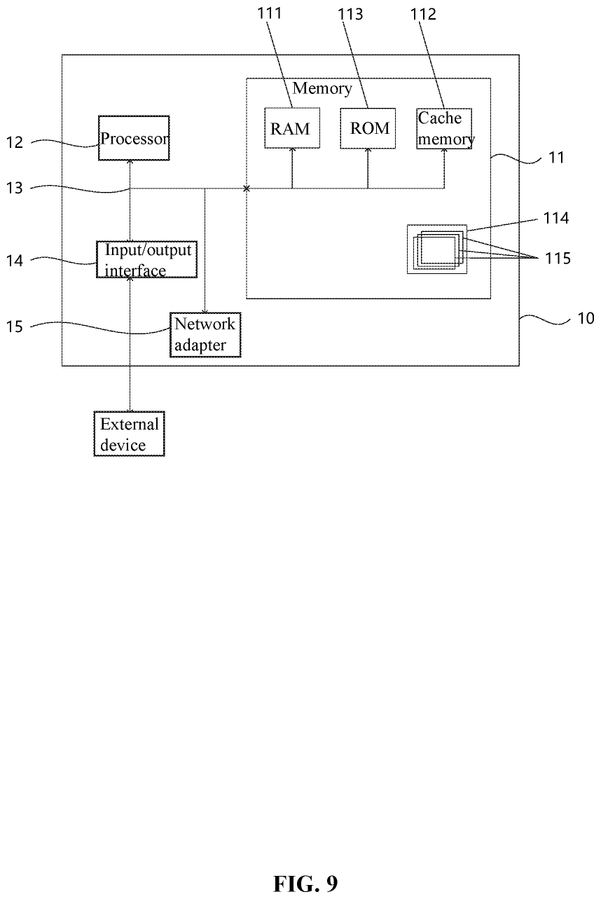

FIG. 9 is a block diagram of a structure of an electronic device according to some embodiments of the present disclosure

As shown in FIG. 9, the electronic device 10 may include, for example, one or more memories 11, one or more processors 12, and a bus 13 connecting different platform systems.

The memory 11 may include a (computer) readable medium in a form of a volatile memory, such as a Random Access Memory (RAM) 111 and/or a cache memory 112. The memory 11 may further include a Read-Only Memory (ROM) 113. The memory 11 further stores a computer program. The computer program may be executed by the processor 12, causing the processor 12 to implement steps of any of the methods described above. The memory 11 may further include a utility 114 having at least one program module 115. The at least one program module 115 includes, but is not limited to, an operating system, one or more applications, other program modules, and program data. Each of these examples or some combination thereof may include an implementation of a network environment.

Accordingly, the processor 12 may execute the computer program and may execute the utility 114. The processor 12 may employ one or more Application Specific Integrated Circuits (ASICs), Digital Signal Processors (DSPs), Programmable Logic Devices (PLDs), Complex Programmable Logic Devices (CPLDs), Field-Programmable Gate Arrays (FPGAs), or other electronic components.

The bus 13 may represent one or more of several types of bus structures, including a memory bus or a memory controller, a peripheral bus, a graphics acceleration port, a processor, or a local bus using any of a variety of bus architectures.

The electronic device 10 may also communicate with one or more external devices (e.g., a keyboard, a pointing device, a Bluetooth device, etc.). The electronic device 10 may further communicate with one or more devices capable of interacting with the electronic device 10, and/or with any device (e.g., a router, a modem, etc.) that enables the electronic device 10 to communicate with one or more other computing devices. Such communication may be executed through an input/output interface 14. Furthermore, the electronic device 10 may also communicate with one or more networks (e.g., a Local Area Network (LAN), a Wide Area Network (WAN), and/or a public network such as the Internet) through a network adapter 15. The network adapter 15 may communicate with other modules of the electronic device 10 through the bus 13. It should be understood that, although not shown in the figure, other hardware and/or software modules may be used in the combination with the electronic device 10 in practical applications. The hardware and/or software modules include, but are not limited to, microcode, device drivers, redundant processors, external disk drive arrays, Redundant Array of Independent Disks (RAID) systems, tape drives, and data backup storage platforms.

In some embodiments of the present disclosure, an electronic device is further provided. Specific embodiments of the electronic device are consistent with embodiments described in the method embodiments above and achieve the same technical effects. Some content is not repeated here.

The electronic device includes a memory and one or more processors. The memory stores a computer program. The one or more processors are configured to implement the method for plan adjustment of radiation therapy according to other embodiments of the present disclosure when executing the computer program.

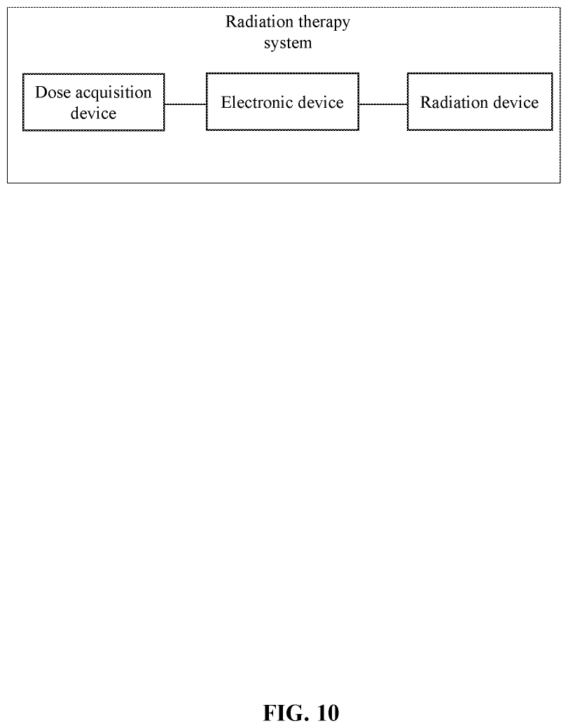

FIG. 10 is a schematic diagram of a structure of a radiation therapy system according to some embodiments of the present disclosure.

As shown in FIG. 10, in some embodiments of the present disclosure, the radiation therapy system is further provided, including:

The electronic device of the device embodiment.

A dose acquisition device configured to acquire surface optical image information of an irradiated region of a patient.

A radiation device is configured to perform particle irradiation on a treatment region of the patient.

The treatment region refers to the target volume and the organ-at-risk region mentioned above. Accordingly, in the radiation therapy system, the electronic device uses any one of the methods for plan adjustment of radiation therapy described above to evaluate a treatment plan and determine whether the treatment plan for a subsequent fractionation of irradiation needs to be adjusted. The method for plan adjustment may involve steps such as dose measurement, optical measurement, and simulation calculation to ensure quality and accuracy of the adjusted treatment plan.

The dose acquisition device is configured to acquire the surface optical image information of the irradiated region of the patient, so that the electronic device acquires a dose distribution of the irradiated region based on the surface optical image information, simulation CT information and a preset mapping relationship, and acquires irradiation dose information of the target volume and the organ-at-risk region. The radiation device is configured to perform the particle irradiation on the treatment region of the patient according to treatment plan information adjusted by the electronic device.

Accordingly, the electronic device evaluates the treatment plan by implementing the method for plan adjustment of the radiation therapy. The process may include the steps such as the dose measurement, the optical measurement, and the simulation calculation. The purpose is to ensure the quality and the accuracy of the treatment plan. The dose acquisition device is configured to acquire the surface optical image information of the irradiated region of the patient, measuring or recording a distribution of the particle irradiation received by the patient during a treatment process. Using the surface optical image information acquired by the dose acquisition device in combination with the simulation CT information and the preset mapping relationship allows for a more precise understanding of a dose distribution of the target volume and the organ-at-risk region of the patient. The radiation device may perform the particle irradiation on the treatment region of the patient according to the treatment plan information adjusted by the electronic device, ensuring that the particle irradiation is performed according to the latest treatment plan information that has been evaluated and adjusted.

An advantage of this approach is that acquiring the surface optical image information of the patient during the treatment process enables real-time monitoring and evaluation of the quality and the accuracy of the treatment plan. This approach helps improve the accuracy of the treatment and ensures that the patient receives a correct irradiation dose. By real-time monitoring and adjustment, the approach can more effectively respond to the physiological changes of the patient and uncertainties during the treatment process, thereby improving the overall effect of the treatment. A timely adjustment of the treatment plan helps reduce potential risks to the patient and ensures that the next treatment is safe and does not cause excessive impact on normal tissues.

In some embodiments, the radiation therapy system further includes one or more of the following devices:

An optical measurement device is configured to measure Cherenkov radiation emitted by the patient due to the particle irradiation, to obtain body surface optical information of the patient.

In some embodiments, the optical measurement device includes one or more photodetectors. When a plurality of photodetectors is used, a photodetector array may be formed. When a field of view of a portion of the photodetectors is blocked, other photodetectors may still detect the Cherenkov radiation. Since a generation time of the Cherenkov radiation is very short, the Cherenkov radiation may be considered to be generated immediately after a particle radiation source irradiates the patient. Therefore, detection windows of the photodetector may match emission windows of the particle radiation source, i.e., at least partially overlapping, to achieve detection of the Cherenkov radiation. Merely by way of example, a control module may be used to turn on and off a detection function of the photodetector. The photodetector is operated to have a series of off periods identical to the series of off periods of the particle radiation source. For example, an electronic gating signal may be provided to the photodetector, causing detection pulses of the photodetector to open and close to form a series of detection windows. Additionally, a shutter may be used to control the turning on and off of the detection function of the photodetector. The shutter moves to block light from reaching the photodetector between the detection windows, and the shutter moves to allow light, including the Cherenkov radiation, to reach the photodetector during the detection windows.

The switching effect of the shutter may be achieved by electronic, mechanical, optical, or software-based means. In practical applications, the detection window of the photodetector may be controlled by the control module and/or the shutter.

### Computer-Readable Storage Medium Embodiment

In some embodiments of the present disclosure, a computer-readable storage medium is further provided. Specific embodiments of the computer-readable storage medium are consistent with embodiments described in the above method embodiments and achieve the same technical effects. Some content is not repeated here.

The computer-readable storage medium stores a computer program. When the computer program is executed by one or more processors, the computer program implements steps of any one of the above methods or implements functions of any one of the above electronic devices.

The computer-readable medium may be a computer-readable signal medium or a computer-readable storage medium. In embodiments of the present disclosure, the computer-readable storage medium may be any tangible medium that contains or stores a program. The program may be used by or in connection with an instruction execution system, a device, or a component. The computer-readable storage medium may be, for example but not limited to, an electrical, magnetic, optical, electromagnetic, infrared, or semiconductor system, the device, or the component, or any suitable combination of the foregoing. More specific examples (a non-exhaustive list) of the computer-readable storage medium include: an electrical connection having one or more wires, a portable disk, a hard disk, a Random Access Memory (RAM), a Read-Only Memory (ROM), an Erasable Programmable Read-Only Memory (EPROM or flash memory), an optical fiber, a portable Compact Disc Read-Only Memory (CD-ROM), an optical storage device, a magnetic storage device, or any suitable combination of the foregoing.

The computer-readable storage medium may include a propagated data signal in a baseband or as part of a carrier wave, the propagated data signal bearing readable program code. Such a propagated data signal may take many forms, including but not limited to electromagnetic signals, optical signals, or any suitable combination thereof. The computer-readable storage medium may also be any computer-readable medium that can send, propagate, or transmit a program for use by or in connection with the instruction execution system, the device, or the component. The program code contained on the computer-readable storage medium may be transmitted using any appropriate medium, including but not limited to wireless, wireline, optical fiber cable, Radio Frequency (RF), etc., or any suitable combination of the foregoing. The program code for executing operations of the present disclosure may be written in any combination of one or more programming languages, including Java, C++, Python, C#, JavaScript, PHP, Ruby, Swift, Go, Kotlin, etc. The program code may execute entirely on a user computing device, partly on the user device, as a stand-alone software package, partly on the user device and partly on a remote computing device, or entirely on the remote computing device or a server. In situations involving the remote computing device, the remote computing device may be connected to the user device through any type of network, including a Local Area Network (LAN) or a Wide Area Network (WAN), or the remote computing device may be connected to an external computing device (for example, through the Internet using an Internet service provider).

In some embodiments of the present disclosure, a computer program product is provided. The computer program product includes a computer program. When the computer program is executed by one or more processors, the computer program implements steps of any one of the above methods or implements functions of any one of the above electronic devices.

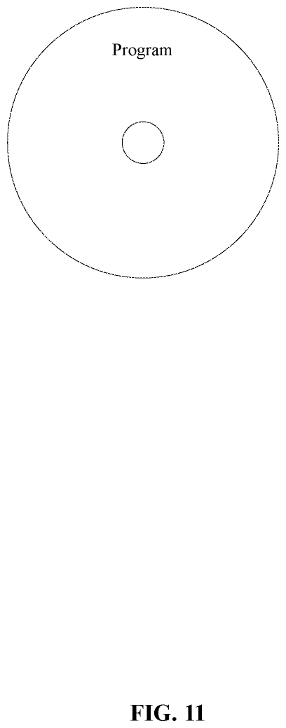

FIG. 11 is a schematic diagram of a structure of a computer program product according to some embodiments of the present disclosure.

As shown in FIG. 11, the computer program product is configured to implement steps of any one of the above methods or implement functions of any one of the above electronic devices. The computer program product may take the form of a portable Compact Disc Read-Only Memory (CD-ROM) and include program code. The computer program product can run on a terminal device, such as a personal computer. However, the computer program product of the present disclosure is not limited thereto. The computer program product may take any combination of one or more computer-readable media.

What is claimed is:

1. A method for plan adjustment of radiation therapy, comprising:
S1, obtaining simulation computed tomography (CT) information and treatment plan information of a patient, wherein the simulation CT information includes a simulation CT image and region segmentation information corresponding to the simulation CT image, the region segmentation information is used to segment a target volume and an organ-at-risk region on the simulation CT image, and the treatment plan information includes a treatment plan parameter of the target volume and the organ-at-risk region;
S2, in a current fractionation of radiation therapy corresponding to the treatment plan information, obtaining surface optical image information of an irradiated region of the patient by measuring Cherenkov radiation emitted from the patient due to particle irradiation;
S3, obtaining a dose distribution of the irradiated region based on the surface optical image information, the simulation CT information, and a preset mapping relationship, and obtaining irradiation dose information of the target volume and the organ-at-risk region based on the dose distribution of the irradiated region;
S4, determining whether the treatment plan information for a subsequent fractionation of irradiation needs to be adjusted based on the irradiation dose information and the treatment plan information; and
S5, in response to determining that the treatment plan information for the subsequent fractionation of irradiation needs to be adjusted, adjusting the treatment plan information for the subsequent fractionation of irradiation based on the dose distribution of the irradiated region and a treatment prescription corresponding to the patient.

2. The method according to claim 1, wherein, before executing S2, the method further includes:
S12, obtaining a detection result of positioning error detection of the patient, and executing S2 when the detection result indicates that positioning of the patient meets a preset positioning condition.

3. The method according to claim 2, wherein the positioning error detection includes image error detection, and the image error detection includes:
obtaining preset positioning information and actual positioning information of the patient;
comparing the actual positioning information with the preset positioning information to obtain a first positioning error value of the image error detection;
when the first positioning error value does not meet a first pass condition, realizing repositioning of the patient by using a positioning device, and re-executing the image error detection; and
when the first positioning error value meets the first pass condition, determining that the positioning of the patient meets the preset positioning condition.

4. The method according to claim 3, wherein, before the image error detection, the positioning error detection further includes laser error detection, and the laser error detection includes:
projecting a laser beam onto the patient, and obtaining an actual projection position of the laser beam;
comparing the actual projection position with a preset projection position to obtain a second positioning error value of the laser error detection; and
when the second positioning error value meets a second pass condition, executing the image error detection.

5. The method according to claim 3, wherein a process for obtaining the actual positioning information includes:
obtaining the actual positioning information by using a radiographic imaging device.

6. The method according to claim 3, wherein the when the first positioning error value does not meet a first pass condition, realizing repositioning of the patient by using a positioning device includes:
generating a positioning adjustment strategy based on the first positioning error value, and controlling the positioning device to execute the positioning adjustment strategy to realize the repositioning of the patient.

7. The method according to claim 1, wherein the obtaining surface optical image information of an irradiated region of the patient by measuring Cherenkov radiation emitted from the patient due to particle irradiation includes:
during the radiation therapy, measuring the Cherenkov radiation emitted from the patient due to the particle irradiation by using an optical measurement device to obtain body surface optical information of the patient;
obtaining an optical-to-dose correspondence between reference body surface optical information and reference body surface dose information, wherein the optical-to-dose correspondence is obtained by using a non-uniform phantom and by measuring the Cherenkov radiation and angular dose deposition in the phantom;
obtaining body surface dose information corresponding to the body surface optical information based on the optical-to-dose correspondence, wherein the body surface dose information corresponding to the body surface optical information is used to indicate a particle dose on a surface of the patient; and
obtaining the surface optical image information of the irradiated region of the patient based on the body surface dose information corresponding to the body surface optical information and the simulation CT image.

8. The method according to claim 7, wherein the method further includes:
determining a physiological characteristic of the irradiated region based on spectral information;
determining whether skin hyperemia exists in the irradiated region based on the physiological characteristic;
in response to determining that the skin hyperemia exists in the irradiated region, generating a correction parameter based on the physiological characteristic; and
correcting the body surface optical information based on the correction parameter.

9. The method according to claim 8, wherein the irradiated region includes a plurality of irradiated points, and the plurality of irradiated points correspond to different correction parameters;
the generating a correction parameter based on the physiological characteristic includes:
determining effective optical pathlengths of the plurality of irradiated points based on expected range depths and camera angles of the plurality of irradiated points;
generating the correction parameters of the plurality of irradiated points based on the physiological characteristic and the effective optical pathlengths of the plurality of irradiated points; and
correcting the body surface optical information based on the correction parameters of the plurality of irradiated points.

10. The method according to claim 8, wherein the determining whether skin hyperemia exists in the irradiated region based on the physiological characteristic includes:
determining an average total blood volume change based on historical physiological characteristics of adjacent regions of the irradiated region;
determining a blood volume offset of the irradiated region based on the physiological characteristic, a baseline total blood volume of the irradiated region, and the average total blood volume change; and
determining whether the skin hyperemia exists in the irradiated region based on the blood volume offset.

11. The method according to claim 10, wherein the generating a correction parameter based on the physiological characteristic includes:
generating the correction parameter based on the blood volume offset and the physiological characteristic.

12. The method according to claim 1, wherein the preset mapping relationship is a correspondence between a secondary electron dose and a total dose obtained after simulating interaction between particles and the patient during treatment by using a Monte Carlo method.

13. The method according to claim 1, wherein the planned treatment parameter includes a prescription dose and a tolerance dose limit;
the determining whether the treatment plan information for a subsequent fractionation of irradiation needs to be adjusted based on the irradiation dose information and the treatment plan information includes:
determining whether the treatment plan information for the subsequent fractionation of irradiation needs to be adjusted based on a difference between the irradiation dose information of the target volume and the prescription dose of the target volume required in the treatment plan information, and a difference between the irradiation dose information of the organ-at-risk region and the tolerance dose limit of the organ-at-risk region.

14. The method according to claim 1, wherein the obtaining simulation CT information of a patient includes:
obtaining the simulation CT image of the patient by using a CT device, and obtaining the simulation CT information according to a contouring operation on the target volume and the organ-at-risk region of the simulation CT image.

15. The method according to claim 1, wherein the adjusting the treatment plan information for the subsequent fractionation of irradiation includes:
inputting the irradiation dose information and the treatment prescription corresponding to the patient into a plan generation model to obtain reference treatment plan information, and updating the treatment plan information of the patient by using the reference treatment plan information.

16. The method according to claim 1, wherein, during the radiation therapy, the method further includes:
comparing the irradiation dose information of the organ-at-risk region with a dose threshold of the current fractionation of radiation therapy; and
when the irradiation dose of the organ-at-risk region exceeds a corresponding dose threshold thereof, generating abnormal prompt information and sending the abnormal prompt information to a user device.

17. The method according to claim 1, wherein, before the determining whether the treatment plan information for a subsequent fractionation of irradiation needs to be adjusted based on the irradiation dose information and the treatment plan information, the method further includes:
determining a target irradiation dose of each of a plurality of future points based on an actual irradiation dose and a standard irradiation dose of each of a plurality of historical points in the irradiated region; and
generating a dose adjustment instruction based on the target irradiation dose of each of the plurality of future points, and sending the dose adjustment instruction to a beam controller, wherein the dose adjustment instruction is configured to control the beam controller to adjust a dwell time of a particle beam pulse at each of the plurality of future points.

18. The method according to claim 17, wherein the method further includes:
in response to determining that the irradiated region is completely scanned, generating a rescanning instruction including a rescanning path and a rescanning dwell time through a path generation model based on a shape feature of the irradiated region, the actual irradiation dose of each of the plurality of historical points, and the standard irradiation dose, wherein the rescanning instruction is configured to control a radiation device to rescan the irradiated region based on the rescanning path, and to stay for the rescanning dwell time at each of a plurality of points on the rescanning path.

19. An electronic device, comprising a memory and one or more processors, wherein the memory stores a computer program, and the one or more processors are configured to implement the following steps when executing the computer program:
S1, obtaining simulation CT information and treatment plan information of a patient, wherein the simulation CT information includes a simulation CT image and region segmentation information corresponding to the simulation CT image, the region segmentation information is used to segment a target volume and an organ-at-risk region on the simulation CT image, and the treatment plan information includes a treatment plan parameter of the target volume and the organ-at-risk region;
S2, in a current fractionation of radiation therapy corresponding to the treatment plan information, obtaining surface optical image information of an irradiated region of the patient by measuring Cherenkov radiation emitted from the patient due to particle irradiation;
S3, obtaining a dose distribution of the irradiated region based on the surface optical image information, the simulation CT information, and a preset mapping relationship, and obtaining irradiation dose information of the target volume and the organ-at-risk region based on the dose distribution of the irradiated region;
S4, determining whether the treatment plan information for a subsequent fractionation of irradiation needs to be adjusted based on the irradiation dose information and the treatment plan information; and
S5, in response to determining that the treatment plan information for the subsequent fractionation of irradiation needs to be adjusted, adjusting the treatment plan information for the subsequent fractionation of irradiation based on the dose distribution of the irradiated region and a treatment prescription corresponding to the patient.

20. A radiation therapy system, comprising:
the electronic device according to claim 19;
a dose acquisition device, configured to acquire surface optical image information of an irradiated region of a patient; and
a radiation device, configured to perform particle irradiation on a treatment region of the patient.
